(19) United States
(12) Patent Application Publication
(10) Pub. No.: US 2021/0077500 A1
(43) Pub. Date: Mar. 18, 2021

(54) PIKFYVE INHIBITORS FOR CANCER THERAPY

(71) Applicant: Al Therapeutics, Inc., Guilford, CT (US)

(72) Inventors: Sean Landrette, Meriden, CT (US); Neil Beeharry, Guilford, CT (US); Peter R. Young, Guilford, CT (US); Jonathan M. Rothberg, Guilford, CT (US); Tian Xu, Shanghai (CN); Henri Lichenstein, Guilford, CT (US)

(21) Appl. No.: 16/948,267

(22) Filed: Sep. 10, 2020

Related U.S. Application Data
(60) Provisional application No. 62/899,392, filed on Sep. 12, 2019.

(51) Int. Cl. A61K 31/5377; A61P 35/04; A61K 31/4184; A61K 31/4545; C12Q 1/686

(52) U.S. Cl. CPC A61K 31/5377; A61P 35/04; C12Q 2563/107; A61K 31/4545; C12Q 1/686; A61K 31/4184

(57) ABSTRACT

The present disclosure relates methods for treating a cancer having activated MET or RAS pathway signaling using an inhibitor of PIKfyve, alone or in combination with a MET inhibitor or a RAS pathway inhibitor, and related compositions and methods to identify PIKfyve inhibitor sensitive cancers for targeted treatment.

### CROSS-REFERENCE TO RELATED APPLICATIONS

This application claims the benefit of priority from U.S. Provisional Application Ser. No. 62/899,392 file on Sep. 12, 2019, the contents of which are hereby fully incorporated by reference.

### FIELD OF THE DISCLOSURE

The present disclosure relates to compositions and methods for treating cancer using inhibitors of PIKfyve.

### BACKGROUND OF THE DISCLOSURE

Cancer is a disease that is commonly caused by mutations that lead to modified proteins with enhanced activity. This unbalanced activity, often in concert with other cellular changes and/or mutations, can drive tumor growth and survival. In some cases, the adaptive mutations of the cancer cells may render them more sensitive to targeted therapeutic agents. In other cases, the cancer cells may adapt to the effects of a targeted agent by developing alternative mechanisms for proliferation and survival. Despite the development of numerous targeted inhibitors against oncogenic signaling pathways, clinical success has been limited. The present disclosure addresses the need to identify cancers that are sensitive to certain targeted therapies as well as the need for alternative therapies and combination therapies that address the adaptive mechanisms of cancer cells.

### SUMMARY OF THE DISCLOSURE

The disclosure provides methods for treating a cancer associated with activated MET or RAS pathway signaling in a subject in need thereof. In embodiments, the methods comprise administering to the subject a pharmaceutical composition comprising a PIKfyve inhibitor, alone or in combination with a MET inhibitor or a RAS pathway inhibitor. A RAS pathway inhibitor can include a RAS inhibitor, a RAF inhibitor, a MEK inhibitor or an ERK inhibitor. In embodiments, the methods comprise determining, ex vivo, the presence of a biomarker of activated MET or RAS pathway signaling in a biological sample comprising cancer cells from the subject, and administering to the subject whose cancer cells are positive for the biomarker a pharmaceutical composition comprising a PIKfyve inhibitor, alone or in combination with a MET inhibitor or a RAS pathway inhibitor. The disclosure also provides the use of a PIKfyve inhibitor in a method of treating a cancer characterized by activated MET or RAS pathway signaling. The disclosure also provides the use of a PIKfyve inhibitor in combination with a MET inhibitor or an inhibitor of RAS pathway signaling in a method of treating a cancer characterized by activated MET or RAS pathway signaling.

In embodiments, the PIKfyve inhibitor is selected from YM201636, WX8(MLS000543798), NDF(MLS000699212), WWL(MLS000703078), XB6(MLS001167897), XBA(MLS001167909), Vacuolin-1, APY-0201, and apilimod.

In some embodiments, the PIKfyve inhibitor is apilimod, or a pharmaceutically acceptable salt thereof. In embodiments, the pharmaceutically acceptable salt may be selected from a sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salt. In embodiments, the pharmaceutically acceptable salt is selected from the group consisting of chloride, methanesulfonate, fumarate, lactate, maleate, pamoate, phosphate, and tartrate. In embodiments, the pharmaceutically acceptable salt is a dimesylate salt.

In some embodiments of the methods for treating cancer, the cancer is selected from a carcinoma, a sarcoma, or a glioma. In embodiments, the cancer cells contain an activating mutation or amplification in the RAS or MET pathway. In embodiments, the cancer is selected from appendiceal cancer, bladder cancer, brain cancer, breast cancer, cervical cancer, colorectal cancer, esophageal cancer, gastric cancer, gastrointestinal carcinoma, gastrointestinal stromal tumor (GIST), genitourinary cancer, glioma, head and neck cancer, hepatocellular carcinoma, lung cancer, melanoma, mesothelioma, non-small cell lung cancer, ovarian cancer, pancreatic cancer, prostate cancer, renal cell carcinoma, sarcoma, small cell lung cancer, soft tissue sarcoma, testicular cancer, thyroid tumor, and uterine carcinosarcoma.

In embodiments, the carcinoma is selected from adenocarcinoma, basal cell carcinoma, squamous cell carcinoma, transitional cell carcinoma, large cell carcinoma, and melanoma. In embodiments, the carcinoma is selected from a pancreatic ductal adenocarcinoma (PDAC), a colorectal carcinoma, a lung carcinoma, such as a non-small cell lung cancer (NSCLC), a renal carcinoma, a head and neck cancer, such as a head and neck squamous cell carcinoma (HNSCC), a gastric carcinoma (GC), and a hepatocellular carcinoma (HCC).

In embodiments, the sarcoma is a soft tissue sarcoma, such as a gastrointestinal stromal tumor (GIST), or a uterine carcinosarcoma.

In embodiments of the methods of treating any of the foregoing cancers, the pharmaceutical composition comprising a PIKfyve inhibitor may be administered either alone, as monotherapy, or in combination with a MET inhibitor or a RAS pathway inhibitor, as combination therapy. In embodiments of combination therapy, the PIKfyve inhibitor may be administered in the same composition or in a different composition from the MET or RAS pathway inhibitor. In embodiments of combination therapy, the MET inhibitor is selected from crizotinib, capmatinib, tepotinib, AMG337, cabozantinib, savolitinib (AZD6094, HMPL-504), tivantinib, foretinib, volitinib, SU11274, PHA 665752, SGX523, BAY-853474, KRC-408, T-1840383, MK-2461, BMS-777607, JNJ-38877605, tivantinib (ARQ 197), PF-04217903, MGCD265, BMS-754807, BMS-794833, AMG-458, NVP-BVU972, AMG-208, golvatinib, norcantharidin, S49076, SAR125844, merestinib (LY2801653), onartuzumab, emibetuzumab, SAIT301, ABT-700, DN30, LY3164530, rilotumumab, ficlatuzumab, TAK701, and YYB-101. In embodiments, the MET inhibitor is selected from crizotinib, capmatinib, tepotinib, AMG337, cabozantinib, and savolitinib (AZD6094, HMPL-504). In a preferred embodiment of combination therapy with a MET inhibitor, the cancer cells contain an activating mutation in or amplification of MET. In embodiments, the cancer treated with combination therapy with a MET inhibitor is a carcinoma, a glioma, or a sarcoma. In embodiments, the cancer is a carcinoma. In embodiments, the carcinoma is selected from breast cancer, colorectal cancer, esophageal cancer, gastric cancer, liver cancer, lung cancer, and renal cancer. In embodiments, the carcinoma is selected from lung cancer, gastric cancer, and renal cancer. In embodiments, the lung cancer is a small cell lung cancer (SCLC) or a non-small cell lung cancer (NSCLC). In embodiments, the cancer is a soft tissue sarcoma, such as a gastrointestinal stromal tumor (GIST), or a uterine carcinosarcoma.

In embodiments of combination therapy, the RAS pathway inhibitor is selected from BVD-523, GDC-0994, binimetinib, cobimetinib, regorafenib, selumetinib, trametinib, vemurafenib, ARS1620, AMG510, AZD4785, MRTX1257, MRTX849, PD-0325901, dabrafenib, encorafenib, pimasertib, and sorafenib. In embodiments, the RAS pathway inhibitor is selected from BVD-523, GDC-0994, trametinib, cobimetinib, binimetinib, selumetinib, regorafenib and vemurafenib. In a preferred embodiment of combination therapy with a RAS inhibitor, the cancer cells contain an activating mutation in the RAS pathway. In embodiments, the cancer treated with combination therapy with a RAS inhibitor is a carcinoma, a glioma, or a sarcoma. In embodiments, the cancer is selected from appendiceal cancer, bladder cancer, brain cancer, breast cancer, cervical cancer, colorectal cancer, esophageal cancer, gastric cancer, gastrointestinal carcinoma, gastrointestinal stromal tumor (GIST), genitourinary cancer, glioma, head and neck cancer, hepatocellular carcinoma, lung cancer, melanoma, mesothelioma, non-small cell lung cancer, ovarian cancer, pancreatic cancer, prostate cancer, renal cell carcinoma, sarcoma, small cell lung cancer, soft tissue sarcoma, testicular cancer, thyroid tumor, and uterine carcinosarcoma. In embodiments, the cancer is a carcinoma selected from bladder cancer, cervical cancer, colorectal cancer, gastric cancer, head and neck squamous cell carcinoma, lung cancer, melanoma, pancreatic cancer, prostate cancer, thyroid cancer, uterine cancer, and urothelial cancer. In embodiments, the cancer is selected from a colorectal cancer, a lung cancer, a melanoma, and a pancreatic cancer. In embodiments, the lung cancer is a small cell lung cancer (SCLC) or a non-small cell lung cancer (NSCLC).

In embodiments, the biomarker of activated MET or RAS pathway signaling is selected from amplification of c-MET, an activating mutation in exon 14 of c-MET, an activating KRAS, NRAS or HRAS mutation and an activating BRAF mutation.

In accordance with any of the foregoing, the cancer may be refractory to standard treatment, or metastatic.

In embodiments, the step of determining, ex vivo, the presence of the biomarker comprises a polymerase chain reaction (PCR)-based assay, 5′exonuclease fluorescence assay, sequencing-by-probe hybridization, dot blotting, oligonucleotide array hybridization analysis, dynamic allele-specific hybridization, molecular beacons, restriction fragment length polymorphism (RFLP)-based methods, flap endonuclease-based methods, primer extension, 5′-nuclease-based methods, oligonucleotide ligase assays, single-stranded conformation polymorphism assays (SSCP), temperature gradient gel electrophoresis, denaturing high performance liquid chromatography (HPLC), high-resolution melting analysis, DNA mismatch-binding methods, capillary electrophoresis, fluorescence in situ hybridization (FISH) and next-generation sequencing (NGS) methods, or a combination of any of the foregoing.

### BRIEF DESCRIPTION OF THE DRAWINGS

FIG. 1: Mutant KRAS is enriched in apilimod-sensitive colorectal cancer cell lines. Sensitive cell lines were LS411N, COLO-205, SW1116, HCT116, SW620, HCT-15, HT29, SW480, and DLD1 and were defined as those having an IC₅₀ less than 200 nM; resistant cell lines were RKO, GP5d, MDST8, SW1417, and COLO-741, defined as those having an IC₅₀ greater than 200 nM. Cell lines having KRAS activating mutations are shown with grey bars.

FIG. 2A-B: Gene expression of KRAS for each of the cell lines indicated in FIG. 1 was obtained from the Cancer Cell Line Encyclopedia (CCLE) database, (A) Affymetrix microarray and (B) RNA seq analysis. Significance was determined by unpaired Student t-test, two-tailed analysis, **p <0.007.

FIG. 3: Apilimod is synergistic with selumetinib in cancer cells with activated RAS pathway. RKO cells were treated with single agent apilimod, selumetinib, or the combination and assayed for viability as described in text. Average cell viability of RKO cells treated with 8 concentrations of apilimod alone (solid line, circles), 8 concentrations of selumetinib alone (solid line, squares) or the combination of apilimod and selumetinib at each single agent dose (dotted line, triangles) is shown. Mean values from two independent experiments are plotted. Shown below the drug concentrations on the x-axis are the average combination index (CI) values. CI values associated with a reduction of cell viability to less than or equal to 25% are shown in bold.

FIG. 4A-B: LS411N (A) or RKO (B) cells were treated with single agent apilimod, selumetinib, or the combination for 120 hours before assaying viability with CellTiter-Glo (Promega). Statistical significance was determined by One-way ANOVA, Dunnett's multiple comparisons test, **** P<0.0001, **P<0.01, *P<0.05. Bars show the average and standard deviation from two independent experiments. The average CI value from the two independent experiments determined by the Chou-Talalay method is also indicated.

FIG. 5A-B: RKO (A) or A549 (B) cells were treated with single agent apilimod, regorafenib, or the combination for 120 hours before assaying viability with CellTiter-Glo (Promega). Statistical significance was determined by One-way ANOVA, Dunnett's multiple comparisons test, **** P<0.0001, ***P<0.001, **P<0.01, *P<0.05. Bars show the average and standard deviation from two independent experiments. The average CI value from the two independent experiments determined by the Chou-Talalay method is also indicated.

FIG. 6A-B: MKN45 (A) or EBC-1 (B) cells were treated with single agent apilimod, and either crizotinib (A) or selumetinib (B) or the combination for 120 hours before assaying viability with CellTiter-GloTM (Promega). Statistical significance was determined by One-way ANOVA, Dunnett's multiple comparisons test, **** P<0.0001, ***P<0.001, **P<0.005. Bars show the average and standard deviation from two independent experiments. The average CI value from the two independent experiments determined by the Chou-Talalay method is also indicated.

### DETAILED DESCRIPTION OF THE DISCLOSURE

The activation of alternative or protective cellular signaling pathways in response to targeted inhibition of signaling by cellular oncogenes such as MET and RAS is highly likely to be a contributing factor in the clinical failure of many single agent targeted therapies. The present invention is based, in part, on the discovery that PIKfyve inhibitors are effective agents for use in cancers characterized by activating mutations in c-MET as well as in cancers characterized by activating mutations in the RAS pathway. The invention is further based, in part, on the discovery that PIKfyve inhibitors are effective agents for use in combination anti-cancer therapy with targeted inhibitors of signaling by the cellular oncogenes MET and RAS, possibly through the ability of the PIKfyve inhibitors to block an escape mechanism, such as autophagy, in the cancer cells. Accordingly, the disclosure provides compositions and methods for use in treating cancers having activating mutations in MET or the RAS pathway with an inhibitor of PIKfyve, either alone, as monotherapy, or in combination with a MET inhibitor and/or a RAS pathway inhibitor. In embodiments, the PIKfyve inhibitor is selected from YM201636, WX8(MLS000543798), NDF(MLS000699212), WWL(MLS000703078), XB6(MLS001167897), XBA(MLS001167909), Vacuolin-1, APY-0201, and apilimod. In embodiments of either monotherapy or combination therapy, the PIKfyve inhibitor is apilimod.

In some embodiments of combination therapy, the PIKfyve inhibitor is apilimod, and the RAS pathway inhibitor is selected from ARS1620, AMG510, MRTX1257, MRTX849, AZD4785, BVD-523, GDC-0994, vemurafenib, dabrafenib, encorafenib, sorafenib, trametinib, cobimetinib, binimetinib, selumetinib, pimasertib, regorafenib and PD-0325901. In some embodiments, the RAS pathway inhibitor is selected from trametinib, cobimetinib, binimetinib, selumetinib, vemurafenib, dabrafenib, encorafenib, sorafenib, regorafenib, BVD-523 and GDC-0994. In some embodiments, the RAS pathway inhibitor is selected from trametinib, cobimetinib, binimetinib, selumetinib, vemurafenib, dabrafenib, encorafenib, sorafenib and regorafenib. In some embodiments, the RAS pathway inhibitor is selected from trametinib, cobimetinib, binimetinib, selumetinib, vemurafenib, regorafenib, BVD-523 and GDC-0994. In one embodiment, the RAS pathway inhibitor is vemurafenib, binimetinib, cobimetinib, selumetinib, trametinib or BVD-523. In embodiments, the MET inhibitor is selected from crizotinib, capmatinib, tepotinib, AMG337, cabozantinib, tivantinib, foretinib, SU11274, PHA 665752, SGX523, BAY-853474, KRC-408, T-1840383, MK-2461, BMS-777607, JNJ-38877605, tivantinib (ARQ 197), PF-04217903, MGCD265, BMS-754807, BMS-794833, AMG-458, NVP-BVU972, savolitinib (AZD6094, HMPL-504), AMG-208, golvatinib, norcantharidin, S49076, SAR125844, merestinib (LY2801653), onartuzumab, emibetuzumab, SAIT301, ABT-700, DN30, LY3164530, rilotumumab, ficlatuzumab, TAK701, and YYB-101. In embodiments, the MET inhibitor is crizotinib, capmatinib, cabozantinib, tepotinib AMG337 and savolitinib. In one embodiment, the MET inhibitor is crizotinib.

PIKfyve is a FYVE-type zinc finger containing kinase, in particular a phosphatidylinositol-3-phosphate 5-kinase. PIKfyve phosphorylates the D-5 position of endosomal phosphatidylinositol and phosphatidylinositol-3-phosphate (PI3P) to respectively yield phosphatidylinositol 5-phosphate (PISP) and phosphatidylinositol 3,5-bisphosphate (PI(3,5)P₂). Phosphoinositides are membrane-bound lipid signaling molecules that regulate multiple biological processes including intracellular signal transduction, organelle transport, cytoskeletal dynamics, ion channel function, and intracellular trafficking. Deregulation of phosphoinositide metabolism is associated with various diseases and disorders including cancer, obesity and diabetes (Balla, T., (2013) Physiol Rev 93, 1019-1137). Inhibition of PIKfyve activity by small molecule inhibitors such as apilimod was shown to cause selective lethality in multiple types of cancer cell lines, and B-cell non-Hodgkin lymphomas were particularly sensitive to apilimod induced cytotoxicity (Gayle et al., (2017) Blood 129, 1768-1778). PIKfyve plays a role in Toll-like receptor signaling, which is important in innate immunity. In immune cells, the selective inhibition of IL-12/IL-23 transcription by apilimod was shown to be mediated by apilimod's direct binding to and inhibition of PIKfyve. See, e.g., Cai et al., (2013) Chem. and Biol. 20, 912-921.

MET is a cell surface receptor tyrosine kinase receptor, also referred to as the hepatocyte growth factor (HGF) receptor, after its ligand, HGF. Amplification of the cellular MET gene, termed “c-MET”, occurs in multiple types of cancers, including carcinomas such as non-small cell lung cancer (NSCLC), gastric cancer (GC), hepatocellular carcinoma (HCC) and glioblastoma. In addition, activating mutations in exon 14 of c-MET are often found in NSCLC (Mo et al., (2017) Chronic Dis. Transl. Med. 3(3):148-153. doi: 10.1016/j.cdtm.2017.06.002; Hu et al., (2018) Cell 175(6), 1665-1678; Pilotto et al., (2017) Ann. Transl. Med. 5(1), 2 doi: 10.21037/atm.2016.12.33. c-MET gene amplification or mutational activation leads to constitutively active intracellular signaling through various pathways including phosphoinositol 3 kinase (PI3K), RAS/MAPK, STAT and WNT pathways. Although a number of targeted MET inhibitors have been developed, specifically protein tyrosine kinase inhibitors and anti-MET receptor or anti-HGF antibodies, they have suffered from a high rate of clinical failure (Inokuchi et al., (2015) World Gastrointest. Oncol. 7(11):317-27; Anestis et al., (2018) Ann. Transl. Med. 6(12), 247; Kim et al., (2017) Exp. Mol. Med. 49(3), e307. doi: 10.1038/emm.2017.17; Miranda et al., (2018) Cancers 10, 280 doi:10.3390/cancers10090280; Mo et al., (2017) Chronic Dis. Transl. Med. 3(3):148-153. doi: 10.1016/j.cdtm.2017.06.002; Rehman et al. (2019) Eur. Med. J. 6(1), 100-111.

As one of the first oncogenes discovered, RAS and its downstream signaling pathway involving RAF, MEK and ERK kinases has been recognized as a major driver of tumor growth. There are three RAS proteins (KRAS, NRAS and HRAS) that harbor GTPase activity and transmit signals from extracellular receptors to internal pathways that direct cell growth, differentiation and survival. While in normal cells RAS proteins alternate between active GTP bound and inactive GDP bound conformations, mutations found in cancer cells lock the RAS proteins in the active form, driving cancer growth. Activating mutations in RAS have been detected in multiple cancers (Hobbs et al. (2016) J. Cell Sci. 129(7), 1287-1292; Dorard et al., (2017) Biochem. Soc. Trans. 45(1), 27-36). Despite decades of research, only recently have direct inhibitors of RAS entered clinical trials, these being in the form of inhibitors directed against the G12C KRAS mutant, and designated ARS1620, AMG510, MRTX1257, MRTX849. See Janes et al., (2018) Cell 172(3), 578-589; Ni et al., (2019) Pharmacol. Thera doi: 10.1016/j.pharmthera.2019.06.007. Other modalities have included antisense (e.g., AZD4785). Activated RAS leads to activation of the downstream RAF-MEK-ERK pathway, each of which has also been targeted by small molecule inhibitors. Mutations in some of these RAS pathway proteins are found in various cancers and can act independently of RAS as cancer drivers. For example, BRAF mutations are frequently found in melanoma, thyroid and colon cancers and MEK mutations in lung and ovarian cancers (Dorard et al., 2017; Gao et al., 2018). Inhibitors for RAF (vemurafenib, dabrafenib, encorafenib, sorafenib, regorafenib), MEK (trametinib, cobimetinib, binimetinib, selumetinib) and ERK/MAPK (BVD-523, GDC-0994) have been developed and tested clinically and are currently approved for some cancers (e.g. BRAF and MEK inhibitors for melanoma) (Kidger et al., (2018) Pharmacol. Thera. doi: 10.1016/j.phamthera.2018. 02.007; Ryan et al., (2015) Trends Cancer 1(3), 183-198; Yaeger et al., (2019) Cancer Discov. 9(3), 329-341. However, despite having a high prevalence of activating RAS mutations, pancreatic ductal adenocarcinoma (PDAC) and colorectal cancers have remained resistant to targeted inhibitors of the RAS signaling pathway (Lee et al., (2019) PNAS USA doi:10.173/pnas.1817494116; Pant et al., (2018) Crit. Rev. Oncol. Hematol. 130, 78-91).

A possible explanation for the disappointing clinical response to MET inhibitors and RAS pathway inhibitors is the concomitant activation by these inhibitors of other signaling pathways that confer resistance. For example, activation of autophagy was reported in MET-amplified gastric cancer cells after treatment with MET tyrosine kinase inhibitors. Lin et al., (2019) Cell Death and Disease 10, 139. Autophagy is considered to be a pro-survival mechanism in many cancers. Consistent with this, inhibiting autophagy enhanced the anti-tumor activity of the MET tyrosine kinase inhibitors in the same cells. Id. A similar induction of autophagy in the presence of RAS pathway inhibitors and enhanced anti-cancer activity from the combination of a RAS pathway inhibitor and an inhibitor of autophagy was seen in cancers having activating RAS mutations. See e.g., Guo et al., (2011) Genes Dev. 25(5), 460-70; Bryant et al., (2019) Nature Med. 25, 628-640; Kinsey et al., (2019) Nature Med. 25, 620-627; Lee et al., (2019) PNAS 110, 4508-4517; White E., (2019) PNAS Comment 116(10), 3965-3967; Seton-Rogers, (2019) PNAS Comments 116(10), 3965-3967.

The present invention extends this work by providing evidence that cancers having activated MET or RAS pathway signaling, for example cancers having amplified c-MET, activating MET mutations, or activating RAS pathway mutations, are preferentially sensitive to PIKfyve inhibitors. Accordingly, the disclosure provides methods of treating cancers having such mutations with inhibitors of PIKfyve, as well as related methods of identifying PIKfyve sensitive cancers for therapy with PIKfyve inhibitors. In addition, the disclosure further provides additional agents for use in combination therapy with MET inhibitors and RAS pathway inhibitors in the form of PIKfyve inhibitors, which the inventors have found to act synergistically when administered in combination with MET inhibitors and/or RAS pathway inhibitors in cancers having amplified MET or activating RAS pathway mutations. In embodiments of the monotherapy and combination therapy regimens described here, the PIKfyve inhibitor is selected from YM201636, WX8(MLS000543798), NDF(MLS000699212), WWL(MLS000703078), XB6(MLS001167897), XBA(MLS001167909), Vacuolin-1, APY-0201, and apilimod. In certain preferred embodiments of the monotherapy and combination therapy regimens described here, the PIKfyve inhibitor is apilimod.

The disclosure provides compositions and methods related to the use of PIKfyve inhibitors for treating cancer in a subject in need of such treatment. The disclosure is based, in part, on the discovery that cancer cells characterized by activated MET or RAS pathway signaling are particularly sensitive to the cytotoxic activity of PIKfyve inhibitors. Accordingly, the disclosure provides methods for targeting PIKfyve inhibitor treatment to patients having cancers characterized by activated MET or RAS pathway signaling, in which such treatment is likely to be most effective, due at least in part to the sensitivity of the cancer cells to the cytotoxic effects of PIKfyve inhibitors. Detection of the presence of one or more of the biomarkers disclosed, i.e. , detection of activating mutations in the RAS signaling pathway, amplification of c-MET, or activating mutations in exon 14 of c-MET, provides a basis for selecting patients for treatment with PIKfyve inhibitors, either as monotherapy or in combination with a targeted inhibitor of MET or RAS pathway signaling. Thus, the present disclosure generally relates to the use of PIKfyve inhibitors to treat patients having cancer cells characterized by one or more biomarkers of activated MET or RAS pathway signaling, and methods for identifying such sensitive cancers by detecting the presence of the one or more biomarkers in cells of the cancer.

The disclosure also provides methods for treating cancer with PIKfyve inhibitor therapy, either as monotherapy or in combination with a targeted inhibitor of MET or RAS pathway signaling, in a subject in need of such treatment, where the subject in need is defined as one whose cancer is characterized by one or more biomarkers of activated MET or RAS pathway signaling. The disclosure also provides methods for identifying a cancer that is sensitive to PIKfyve inhibitor therapy, the methods comprising assaying for one or more of the biomarkers. In embodiments, the methods may comprise obtaining a biological sample comprising cancer cells from the subject, and assaying for the presence of the one or more biomarkers in the cancer cells. In embodiments, the methods may comprise administering a PIKfyve inhibitor, either alone as monotherapy or in combination with a targeted inhibitor of MET or RAS pathway signaling, to treat cancer in a subject where one or more of the biomarkers is detected in a biological sample of cancer cells obtained from the subject.

In certain embodiments of the methods described here, one PIKfyve inhibitor is apilimod. As used herein, the term “apilimod” refers to apilimod free base, but the compositions and methods described here also encompass pharmaceutically acceptable salts of apilimod, including for example apilimod dimesylate, and other salts as described below. The structure of apilimod is shown in Formula I:

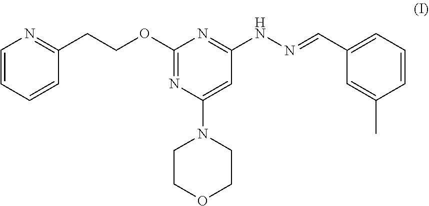 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0. Apilimod can be prepared, for example, according to the methods described in U.S. Pat. Nos. 7,923,557, and 7,863,270, and WO 2006/128129.

Based upon its activity as an immunomodulatory agent and a specific inhibitor of IL-12/IL-23, apilimod, also referred to as STA-5326, was initially proposed as useful in treating autoimmune and inflammatory diseases and disorders. See e.g., Wada et al. (2007) Blood 109, 1156-1164; and U.S. Pat. Nos. 6,858,606 and 6,660,733 (describing a family of pyrimidine compounds, including apilimod, purportedly useful for treating diseases and disorders characterized by IL-12 or IL-23 overproduction, such as rheumatoid arthritis, sepsis, Crohn's disease, multiple sclerosis, psoriasis, or insulin dependent diabetes mellitus). Similarly, apilimod was suggested to be useful for treating certain cancers based upon its activity to inhibit c-Rel or IL-12/23, particularly in cancers where these cytokines were believed to play a role in promoting aberrant cell proliferation. See e.g., WO 2006/128129 and Baird et al., (2013) Frontiers in Oncology 3, 1, respectively.

As used herein, the term “pharmaceutically acceptable salt,” is a salt formed from, for example, an acid and a basic group of apilimod. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts.

In some embodiments, the salt form of apilimod is a methanesulfonate, for example apilimod dimesylate. In some embodiments, the salt form is selected from a glycolate, hemi-fumarate, hydrochloride, DL-lactate, maleate, malonate, phosphate, and L-tartrate. Additional illustrative salts include acetate, acid citrate, acid phosphate, ascorbate, benzoate, benzenesulfonate, bisulfate, bitartrate, bromide, besylate, chloride, citrate, ethanesulfonate, fumarate, formate, gentisinate, glucaronate, gluconate, glutamate, iodide, isonicotinate, lactate, maleate, nitrate, oleate, oxalate, pantothenate, p-toluenesulfonate, pamoate (e.g., 1,1′-methylene-bis-(2-hydroxy-3-naphthoate)), saccharate, salicylate, succinate, sulfate, tannate, and tartrate.

The term “pharmaceutically acceptable salt” also refers to a salt prepared from an apilimod composition having an acidic functional group, such as a carboxylic acid functional group, and a pharmaceutically acceptable inorganic or organic base.

The term “pharmaceutically acceptable salt” also refers to a salt prepared from apilimod having a basic functional group, such as an amino functional group, and a pharmaceutically acceptable inorganic or organic acid.

### Methods of Treatment

The present disclosure provides methods for treating a cancer associated with activated MET or RAS pathway signaling with a PIKfyve inhibitor, either alone or in combination with a MET inhibitor or a RAS pathway inhibitor, and related compositions and methods. For example, the disclosure also provides methods of identifying a cancer that is sensitive to monotherapy or combination therapy with a PIKfyve inhibitor by detecting a biomarker of MET or RAS pathway activation in a biological sample of the cancer. In the context of the methods described here, MET or RAS pathway ‘activation’ or an ‘activated’ MET or RAS pathway refers to signaling by the pathway that is decoupled from, or outside of, the normal cellular controls that regulate both the initial activation of the signaling pathway and the duration of its signaling. For example, in some embodiments, a cancer associated with “activated” MET or RAS pathway signaling may be one in which the pathway is constitutively active. Such constitutive activity may also be refered to as “ligand-independent” activation to signify its decoupling from the ligand-receptor interaction that is typically required for MET or RAS pathway activation. Constitutive activation of a MET or RAS pathway may also occur by various mechanisms, for example, certain mutations of the Ras GTPase that prevent GTP hydrolysis, thereby preventing the regulated termination of the signal and resulting in its contitutive activation. Such constitutive activation of the MET or RAS pathway outside of the normal cellular control mechanisms may also be referred to as “oncogenic activation” to the extent it plays a role, or is likely to play a role, in driving cancer cell proliferation and/or survival. Accordingly, in some embodiments, a cancer treated according to the present methods may be referred to as a cancer associated with oncogenic activation of the MET or RAS pathway. There are numerous mechanisms through which MET or RAS pathway signaling may become activated in cancer, resulting in its dysregulation from normal cellular controls and constitutive activation. For example, such activation may result from mutations in one or more genes or proteins of the pathways or amplification of one or more genes or proteins in the pathway. Examples of such genetic mutations and amplifications are provided infra. In addition, other mechanisms whereby the pathway is activated and/or remains in an active state independently from normal cellular controls. For example, pathway activation may occur via aberrant transcriptional activation or alternative splicing, aberrant translational control, aberrant phosphorylation, aberrant proteolysis, aberrant subcellular localization, etc., as well as epigenetic mechanisms including histone-tail modifications, DNA methylation, chromatin remodeling, and regulation of non-coding RNA expression such as microRNAs (miRNA). These and other mechanisms of aberrant RAS and MET pathway activation in the context of cancer are described in Stephens et al., 2017 Cancer Informatics 16: 1-10; Masliah-Planchon et al., 2015 Oncotarget 7(25): 38892; and Zhang and Babic, Carcinogenesis 2016 37(4):345.

In some embodiments, the methods described here comprise assaying a biological sample of cancer cells obtained from the subject to detect the presence of a biomarker of activated MET or RAS pathway signaling in the cancer cells. Examples of suitable biomarkers are described infra.

In embodiments, the disclosure provides methods for treating a cancer in a subject in need thereof, the methods comprising determining, ex vivo, the presence of one or more biomarkers of activated MET or RAS pathway signaling in a biological sample comprising cancer cells from the subject, and administering to the subject a pharmaceutical composition comprising a PIKfyve inhibitor where the one or more biomarkers is determined to be present in the cancer cells.

In embodiments, the disclosure also provides methods for identifying a subject having a cancer that is susceptible to treatment with a PIKfyve inhibitor, the methods comprising detecting the presence of one or more biomarkers of activated MET or RAS pathway signaling in a biological sample comprising cancer cells of the subject.

In embodiments, the disclosure also provides methods for selecting a treatment for a subject having a cancer, the methods comprising determining, ex vivo, the presence of one or more biomarkers of activated MET or RAS pathway signaling in a biological sample comprising cancer cells from the subject, and selecting a PIKfyve inhibitor for treatment of the subject where the one or more biomarkers is determined to be present in the cancer cells.

In embodiments, the disclosure also provides methods for predicting the efficacy of a PIKfyve inhibitor in a therapeutic regimen for treating a cancer in a subject, the methods comprising determining, ex vivo, the presence of one or more biomarkers of activated MET or RAS pathway signaling in a biological sample comprising cancer cells from the subject, and predicting that the subject may be effectively treated with a therapeutic regimen comprising a PIKfyve inhibitor where the one or more biomarkers is determined to be present in the cancer cells.

In accordance with the embodiments described here, a PIKfyve inhibitor is administered to the subject having a cancer characterized by activated MET or RAS pathway signaling, for example as determined by the presence of a biomarker of activated MET or RAS pathway signaling in a biological sample comprising cancer cells from the subject. In some embodiments, the PIKfyve inhibitor is administered as monotherapy. In some embodiments, the PIKfyve inhibitor is administered as part of a therapeutic regimen, in combination with a MET inhibitor or a RAS pathway inhibitor. In accordance with either the monotherapy or combination therapy embodiments, the PIKfyve inhibitor may be selected from YM201636, WX8(MLS000543798), NDF(MLS000699212), WWL(MLS000703078), XB6(MLS001167897), XBA(MLS001167909), Vacuolin-1, APY-0201, and apilimod. In certain preferred embodiments, the PIKfyve inhibitor is apilimod, or a pharmaceutically acceptable salt thereof. In one embodiment, the salt form is the dimesylate salt.

The disclosure provides methods for treating cancers characterized by activated MET or RAS pathway signaling with a PIKfyve inhibitor. Such cancers may be characterized using a biomarker of activated MET or RAS pathway signaling. In embodiments, the biomarker of activated MET or RAS pathway signaling is a mutation selected from amplification of c-MET, an activating mutation in exon 14 of c-MET, an activating KRAS, NRAS or HRAS mutation, and an activating BRAF mutation. In some embodiments, the activating KRAS mutations is selected from KRAS G12(V,C,S,R,D,N,A), G13(D,C,R), Q22K, Q61(H,L,R), K117N and A146(TN) where the letter designations refer to the one-letter amino acid symbols recommended by the IUPAC-IUB Biochemical Nomenclature Commission. In some embodiments, the activating NRAS mutations is selected from NRAS G12(V,C,S, D,N,A), G13(D,C,V,R,A,S), Q61(H,L,R,K,P). In some embodiments, the activating HRAS mutations is selected from HRAS G12(V,C,S,R,D,N,A), G13(R,V,D,S,C), Q61(K,L,R,H). In some embodiments, the activating KRAS mutation is selected from KRAS G12S, G12D, and G12C. In some embodiments, the activating BRAF mutation is V600E or V600K. In some embodiments, the activating BRAF mutation is an oncogenic deletion mutation, for example DNVTAP, DTAPTP, or DPTPQQ. In some embodiments, the activating BRAF mutation is a gene rearrangement.

In embodiments, the cancer treated according to the methods described here is a carcinoma, a glioma, or a sarcoma. In embodiments, the cancer is not a leukemia, lymphoma, or myeloma. In some embodiments where the cancer is a carcinoma, the carcinoma is selected from adenocarcinoma, basal cell carcinoma, squamous cell carcinoma, transitional cell carcinoma, large cell carcinoma, and melanoma. In some embodiments, the carcinoma, glioma, or sarcoma is characterized by one or more activating RAS pathway mutations or one or more activating MET pathway mutations.

In some embodiments, the cancer treated according to the methods described here is selected from appendiceal cancer, bladder cancer, brain cancer, breast cancer, cervical cancer, colorectal cancer, esophageal cancer, gastric cancer, gastrointestinal carcinoma, gastrointestinal stromal tumor (GIST), genitourinary cancer, glioma, head and neck cancer, hepatocellular carcinoma, lung cancer, melanoma, mesothelioma, non-small cell lung cancer, ovarian cancer, pancreatic cancer, prostate cancer, renal cell carcinoma, sarcoma, small cell lung cancer, soft tissue sarcoma, testicular cancer, thyroid tumor, and uterine carcinosarcoma. In embodiments, the cancer is a lung cancer, preferably a non-small cell lung cancer (NSCLC).

In embodiments where the cancer cells carry one or more activating RAS pathway mutations and the cancer is a carcinoma, the carcinoma may be selected from bladder cancer, cervical cancer, colorectal cancer, gastric cancer, head and neck squamous cell carcinoma, lung cancer, melanoma, pancreatic cancer, prostate cancer, thyroid cancer, uterine cancer, and urothelial cancer. In some embodiments, the carcinoma is selected from a colon carcinoma, a lung carcinoma, such as a non-small cell lung cancer (NSCLC), a melanoma, and a head and neck cancer, such as a head and neck squamous cell carcinoma (HNSCC), and a pancreatic ductal adenocarcinoma (PDAC).

In some embodiments where the cancer cells carry one or more activating RAS pathway mutations, the cancer is selected from a melanoma, a colorectal cancer, thyroid cancer, or lung cancer with an activating mutation in BRAF. In some embodiments, the BRAF mutation is a class I mutation of V600 to E/K/D/R or a class II mutation at R462, I463, G464, G469, E586, F595, L597, A598, T599, K601, A727, or a BRAF fusion (See e.g., Dankner et al., (2018) Oncogene 37(24): 3183-3199. In some embodiments the cancer is melanoma, a prostate cancer, or a gastric cancer with an activating gene rearrangement in BRAF or RAF-1 (See e.g., Palanisamy et al., (2010) Nature Med. 16(7): 793-798.

In some embodiments where the cancer cells carry one or more activating RAS pathway mutations, the cancer is selected from a cervical, colorectal, gastric lung, pancreatic, or uterine cancer with an activating mutation in the KRAS gene. In some embodiments, the cancer is acute myeloid leukemia or multiple myeloma with an activating mutation in the KRAS gene. In some embodiments, the activating KRAS mutation is selected from a mutation in the codon for an amino acid selected from G12, G13, Q22, Q61, K117 and A146 of KRAS, as described in Hobbs et al., (2016) Cancer Cell 29(3), 251-253 and Edkins et al., (2006) Cancer Biol. Thera. 5(8), 928-932.

In some embodiments where the cancer cells carry one or more activating RAS pathway mutations, the cancer is selected from a bladder cancer, a colorectal cancer, a head and neck squamous cell carcinoma, a lung cancer, a melanoma, or a thyroid cancer with an activating mutation in NRAS. In some embodiments, the cancer is acute myeloid leukemia with an activating mutation in NRAS. In embodiments, the activating mutation in NRAS is selected from a mutation in the codon for an amino acid selected from G12, G13, Q61 and A146 as described in Hobbs and Edkins above.

In some embodiments where the cancer cells carry one or more activating RAS pathway mutations, the cancer is selected from a head and neck squamous cell carcinoma, melanoma or urothelial cancer with an activating mutation in HRAS. In some embodiments, the cancer is acute myeloid leukemia with an activating mutation in HRAS. In embodiments, the activating mutation is selected from a mutation in the codon for an amino acid selected from G12, G13, Q61 and A146 as described in Hobbs et al., (2016) Cancer Cell 29(3), 251-253; Edkins et al., (2006) Cancer Biol. Thera. 5(8), 928-932.

In embodiments where the cancer cells carry one or more activating MET pathway mutations, the cancer may be selected from breast cancer, colorectal cancer, esophageal cancer, gastric cancer, glioma, liver cancer, lung cancer, or renal cancer with an amplification of the c-MET gene and/or increased expression or activity of c-MET protein. In some embodiments, the mutation is amplified c-MET or an activating MET mutation, and the cancer is a carcinoma selected from NSCLC, gastric cancer (GC), hepatocellular carcinoma (HCC) and glioblastoma. In some embodiments, the cancer is a lung cancer or glioma with an activating mutation in exon 14 of c-MET. Activating mutations may include base substitutions and indels (insertions or deletions) at splice acceptor or donor sites or in the ˜25 bp intronic noncoding region immediately adjacent to the splice acceptor site, or indels resulting in the entire deletion of exon 14 (Frampton et al., (2015) Cancer Discov. 5(8), 850-859). In some embodiments, the cancer is a liver, lung, gastric, GIST (gastrointestinal stromal tumor), renal, breast, colorectal cancer with a mutation in the kinase, juxtamembrane or Sema domains of c-MET. Further activating MET mutations include substitutions in the DNA encoding the following amino acids: E34, H150 E168, L269, L299, S323, M362, N375, C385 (Sema domain); R970, R988, P1009, T1010, S1058 (juxtamembrane domain), A1108, V1110, H1112, H1124, G1137, M1149, T1191, V1206, L1213, D1228, Y1230, Y1235, V1238, D1246, Y1248, K1262, M1268 and V1312 (Tovar et al., (2017) Ann. Transl. Med. 5(10), 205-210).

The disclosure also provides methods for identifying a cancer that is sensitive to a PIKfyve inhibitor, the methods comprising assaying for the presence of one or more biomarkers of activated MET or RAS pathway signaling, for example one or more activating mutations in MET, RAS or BRAF as discussed above, and/or increased expression of MET, RAS or BRAF in the cancer cells. In embodiments, the methods may comprise obtaining a biological sample comprising cancer cells from the subject and assaying for the presence of one or more activating mutations in MET, RAS or BRAF and/or increased expression of MET, RAS or BRAF in the cancer cells. In embodiments, the methods may further comprise administering a PIKfyve inhibitor to treat a cancer in a subject where the presence of one or more biomarkers of activated MET or RAS pathway signaling is detected in a biological sample of cancer cells obtained from the subject.

In some embodiments, where the methods described here include determining the presence of a biomarker of activated MET or RAS pathway signaling, determining the presence of the biomarker may include a step of detecting one or more nucleotide or amino acid variants of c-MET, HGF, KRAS, NRAS, or BRAF, or their encoded proteins, MET, HGF, KRAS, NRAS, HRAS and BRAF. Where the variant is in an exon of a gene encoding a protein, the variant may be detected either in the genomic DNA or in the RNA of the cancer cells. The variant may also include increased copy number of exons in the genes c-MET, HGF, KRAS, NRAS, HRAS, or BRAF.

In embodiments, the methods may comprise determining the subject's genotype to detect the presence of a biomarker of activated MET or RAS pathway signaling. The genotype may be determined by techniques known in the art, for example, PCR-based methods, DNA sequencing, 5′exonuclease fluorescence assay, sequencing by probe hybridization, dot blotting, and oligonucleotide array hybridization analysis, for example, high-throughput or low density array technologies (also referred to as microarrays and gene chips), and combinations thereof. Other specific techniques may include dynamic allele-specific hybridization, molecular beacons, restriction fragment length polymorphism (RFLP)-based methods, flap endonuclease-based methods, primer extension, 5′-nuclease-based methods, oligonucleotide ligase assays, single-stranded conformation polymorphism assays (SSCP), temperature gradient gel electrophoresis, denaturing high performance liquid chromatography (HPLC), high-resolution melting analysis, DNA mismatch-binding methods, capillary electrophoresis, fluorescence in situ hybridization (FISH), and next-generation sequencing (NGS) methods. Real-time PCR methods that can be used to detect SNPs, include, e.g., Taqman or molecular beacon-based assays (U.S. Pat. Nos. 5,210,015; 5,487,972; and PCT WO 95/13399). Genotyping technology is also commercially available, for example from companies such as Applied Biosystems, Inc. (Foster City, Calif.).

In embodiments, genotype may be determined by a method selected from direct manual sequencing, automated fluorescent sequencing, single-stranded conformation polymorphism assays (SSCPs), clamped denaturing gel electrophoresis (CDGE), denaturing gradient gel electrophoresis (DGGE), mobility shift analysis, restriction enzyme analysis, heteroduplex analysis, chemical mismatch cleavage (CMC), and RNase protection assays.

In embodiments, the method of detecting the presence of a biomarker may comprise a step of contacting a set of SNP-specific primers with DNA extracted from a sample of cancer cells from the subject, allowing the primers to bind to the DNA, and amplifying the SNP containing regions of the DNA using a polymerase chain reaction.

In embodiments, the methods described here may comprise receiving, in a computer system, the patient's genotype for a biomarker described here. In one embodiment, a user enters the patient's genotype in the computer system. In one embodiment, the patient's genotype is received directly from equipment used in determining the patient's genotype.

In some embodiments, the biomarker may be a marker of gene expression, for example mRNA or protein abundance, e.g., overexpression of MET or RAS or activation of downstream signaling molecules (induction or changes in post-translational modifications such as phosphorylation). Suitable methods for detecting gene expression include methods comprising microarray expression analysis, PCR-based methods, in situ hybridization, Northern immunoblotting and related probe hybridization techniques, single molecule imaging technologies such as nCounter® or next generation sequencing methods such as RNA-seg™ (Life Technologies) and SAGE technologies™ and combinations of the foregoing. In embodiments, the methods may comprise detection of protein expression or post-translational modification (e.g. phosphorylation) using a suitable method comprising one or more of immunohistochemistry, mass spectrophotometry, flow cytometry, an enzyme-linked immunoabsorbant assay, Western immunoblotting and related probe hybridization techniques, multiplex immunoassay (e.g., Luminex®, MesoScale™ Discovery, SIMOA™), single molecule imaging technologies such as nCounter®, and aptamer-based multiplex proteomic technologies such as SOMAscan®.

In embodiments, the methods may further comprise obtaining a biological sample of cancer cells from the subject in need of treatment, for example by a biopsy procedure. In this context, a biopsy procedure comprises extracting a sample of cancer cells or tissue comprising cancer cells from the subject. The biopsy may be performed, for example, as an incisional biopsy, a core biopsy, or an aspiration biopsy, e.g., fine needle aspiration.

### Combination Therapy

The present disclosure also provides methods comprising combination therapy for the treatment of cancer using a PIKfyve inhibitor in combination with a MET inhibitor or a RAS pathway inhibitor, or both. As used herein, “combination therapy” or “co-therapy” includes the administration of a therapeutically effective amount of a PIKfyve inhibitor as part of a specific treatment regimen intended to provide the beneficial effect from the co-action of the PIKfyve inhibitor and the additional agent, in this case the MET inhibitor and/or the RAS pathway inhibitor. The beneficial effect of the combination includes, but is not limited to, pharmacokinetic or pharmacodynamic co-action resulting from the combination of therapeutic compounds. The beneficial effect of the combination may also relate to the mitigation of a toxicity, side effect, or adverse event associated with another agent in the combination. “Combination therapy” is not intended to encompass the administration of two or more of these therapeutic compounds as part of separate monotherapy regimens that incidentally and arbitrarily result in a beneficial effect that was not intended or predicted.

Accordingly, the disclosure also provides PIKfyve inhibitors for use in combination with MET and/or RAS pathway inhibitors for the treatment of cancers having amplified MET or activated RAS pathway signaling. In some embodiments, the PIKfyve inhibitor for use in such combination therapy is selected from the group consisting of YM201636, WX8(MLS000543798), NDF(MLS000699212), WWL(MLS000703078), XB6(MLS001167897), XBA(MLS001167909), Vacuolin-1, APY-0201, and apilimod. In certain preferred embodiments of the combination therapy regimens described here, the PIKfyve inhibitor is apilimod.

In some embodiments, the PIKfyve inhibitor is administered in combination with an inhibitor of MET or an inhibitor of the RAS pathway, or both. In some embodiments, the inhibitor of MET is selected from a small molecule inhibitor of the kinase domain or an inhibitory antibody. In embodiments, the inhibitor of MET is a small molecule kinase inhibitor selected from crizotinib, capmatinib, tepotinib, AMG 337, cabozantinib, tivantinib, foretinib, SU11274, PHA 665752, SGX523, BAY-853474, KRC-408, T-1840383, and MK-2461. In embodiments, the inhibitor of MET is a small molecule kinase inhibitor selected from BMS-777607, JNJ-38877605, tivantinib (ARQ 197), PF-04217903, MGCD265, BMS-754807, BMS-794833, AMG-458, NVP-BVU972, savolitinib(AZD6094, HMPL-504), AMG-208, golvatinib, norcantharidin, S49076, SAR125844, and merestinib (LY2801653). In some embodiments, the inhibitor of MET is an anti-MET receptor antibody or an antibody against the MET ligand HGF, also referred to as an anti-HGF antibody. In embodiments, the anti-MET receptor antibody is selected from onartuzumab, emibetuzumab, SAIT301, ABT-700, DN30, and LY3164530. In embodiments, the anti-HGF antibody is selected from rilotumumab, ficlatuzumab, TAK701, and YYB-101.

In some embodiments, the RAS pathway inhibitor is a targeted inhibitor of RAS, MEK, ERK/MAPK, or RAF. In some embodiments, the RAS pathway inhibitor is a RAS inhibitor selected from ARS1620, AMG510, MRTX1257, MRTX849 and AZD4785. In some embodiments, the RAS pathway inhibitor is an ERK/MAPK inhibitor selected from BVD-523 and GDC-0994. In some embodiments, the RAS pathway inhibitor is a RAF inhibitor selected from vemurafenib, dabrafenib, encorafenib, regorafenib and sorafenib. In some embodiments, the RAS pathway inhibitor is a MEK inhibitor selected from trametinib, cobimetinib, binimetinib, selumetinib, pimasertib, and PD-0325901. In some embodiments, the RAS pathway inhibitor is selumetinib.

In embodiments, where the PIKfyve inhibitor is apilimod, the apilimod may optionally be administered in combination with an agent intended to mitigate one or more side effects of the apilimod, for example one or more of nausea, vomiting, headache, dizziness, lightheadedness, drowsiness and stress. In one aspect of this embodiment, the agent is an antagonist of a serotonin receptor, also known as 5-hydroxytryptamine receptors or 5-HT receptors. In one aspect, the agent is an antagonist of a 5-HT3 or 5-HT1a receptor. In one aspect, the agent is selected from the group consisting of ondansetron, granisetron, dolasetron and palonosetron. In another aspect, the agent is selected from the group consisting of pindolol and risperidone.

In embodiments, the at least one additional API administered in combination therapy with a PIKfyve inhibitor is a RAS pathway inhibitor or a c-MET inhibitor.

In embodiments, the RAS pathway inhibitor is a RAF inhibitor selected from PLX4032 (vemurafenib), PLX-4720 (sorafenib), GSK2118436 (dabrafenib), BAY 73-4506 (regorafenib), GDC-0879, RAF265, AZ 628, NVP-BHG712, SB90885, ZM 336372, GW5074, TAK-632, CEP-32496 and LGX818 (Encorafenib). In one embodiment, the RAF inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a RAF (e.g., A-RAF, B-RAF, C-RAF) or a nucleic acid encoding the RAF protein.

In embodiments, the RAS pathway inhibitor is a MEK inhibitor selected from AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733 and PD318088. In one embodiment, the MEK inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a MEK (e.g., MEK-1, MEK-2) or a nucleic acid encoding the MEK protein.

In embodiments, the RAS pathway inhibitor is an ERK inhibitor selected from BVD-523 and GDC-0994. In one embodiment, the ERK inhibitor is a polypeptide (e.g., an antibody or fragment thereof) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of a ERK (e.g., ERK-1, ERK-2) or a nucleic acid encoding the ERK protein.

In embodiments, the c-MET inhibitor is selected from crizotinib, tivantinib, capmatinib, tepotinib, cabozantinib, foretinib, savolitinib and AMG 337. In one embodiment, the c-MET inhibitor is a polypeptide (e.g., an antibody or fragment thereof, exemplified by onartuzumab) or nucleic acid (e.g., a double-stranded small interfering RNA, a short hairpin RNA, a micro-RNA, an antisense oligonucleotide, a morpholino, a locked nucleic acid, or an aptamer) that binds to and inhibits the expression level or activity of c-MET or a nucleic acid encoding the c-MET protein or the HGF ligand, such as ficlatuzumab or rilotumumab.

“Combination therapy” also embraces the administration of a PIKfyve inhibitor as described here in further combination with non-drug therapies (e.g., surgery or radiation treatment). Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

The non-drug treatment can be selected from chemotherapy, radiation therapy, hormonal therapy, anti-estrogen therapy, gene therapy, surgery (e.g. radical nephrectomy, partial nephrectomy, laparoscopic and robotic surgery), radiofrequency ablation, and cryoablation. For example, a non-drug therapy is the removal of an ovary (e.g., to reduce the level of estrogen in the body), thoracentesis (e.g., to remove fluid from the chest), paracentesis (e.g., to remove fluid from the abdomen), surgery to remove or shrink angiomyolipomas, lung transplantation (and optionally with an antibiotic to prevent infection due to transplantation), or oxygen therapy (e.g., through a nasal cannula containing two small plastic tubes or prongs that are placed in both nostrils, through a face mask that fits over the nose and mouth, or through a small tube inserted into the windpipe through the front of the neck, also called transtracheal oxygen therapy).

In the context of combination therapy, administration of a PIKfyve inhibitor may be simultaneous with or sequential to the administration of the MET inhibitor or the RAS pathway inhibitor. In some embodiments, administration of the different components of a combination therapy may be at different frequencies. For example, one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the initial component administered in the regimen.

In some embodiments, the PIKfyve inhibitor is formulated for co-administration with the MET inhibitor or the RAS pathway inhibitor in a single dosage form. In some embodiments, the PIKfyve inhibitor is administered separately from the dosage form that comprises the MET inhibitor or the RAS pathway inhibitor. When a different agent is administered separately from the PIKfyve inhibitor, it can be administered by the same or a different route of administration as the PIKfyve inhibitor.

Preferably, the administration of a composition comprising a PIKfyve inhibitor in combination with one or more additional active agents, such as a MET inhibitor or the RAS pathway inhibitor, provides a synergistic response in the subject being treated. In this context, the term “synergistic” refers to the efficacy of the combination being more effective than the additive effects of either single therapy alone. The synergistic effect of a combination therapy according to the disclosure can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

In the context of the methods described herein, the amount of a PIKfyve inhibitor administered to the subject is a therapeutically effective amount. The term “therapeutically effective amount” refers to an amount sufficient to treat, ameliorate a symptom of, reduce the severity of, or reduce the duration of the disease or disorder being treated or enhance or improve the therapeutic effect of another therapy, or sufficient to exhibit a detectable therapeutic effect in the subject. In one embodiment, the therapeutically effective amount of an apilimod composition is the amount effective to inhibit PIKfyve kinase activity in cancer cells of the subject.

An effective amount can range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the disease treated, route of administration, excipient usage, and the possibility of co-usage with other therapeutic treatments such as use of other agents.

In more specific aspects, the PIKfyve inhibitor is administered at a dosage regimen of 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, apilimod is administered at a dosage regimen of 100-1000 mg/day for 4 or 16 weeks. Alternatively or subsequently, apilimod is administered at a dosage regimen of 100-300 mg twice a day for 8 weeks, or optionally, for 52 weeks. Alternatively or subsequently, an apilimod composition is administered at a dosage regimen of 50-1000 mg twice a day for 8 weeks, or optionally, for 52 weeks.

An effective amount of the PIKfyve inhibitor can be administered once daily, from two to five times daily, up to two times or up to three times daily, or up to eight times daily. In one embodiment, the PIKfyve inhibitor is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in a 3-week cycle, or once every two days, or once a week, or once every 2 weeks, or once every 3 weeks.

In embodiments of the methods described here, the subject in need of treatment may be one having a cancer that is non-responsive or refractory to, or has relapsed after, treatment with a ‘standard-of-care’ or first-line therapeutic agent. In this context, the terms “non-responsive” and “refractory” are used interchangeably and refer to the subject's response to therapy as not clinically adequate, for example to stabilize or reduce the size of one or more solid tumors, to slow tumor progression, to prevent, reduce or decrease the incidence of new tumor metastases, or to relieve one or more symptoms associated with the cancer. A cancer that is refractory to a particular drug therapy may also be described as a drug resistant cancer. In a standard therapy for the cancer, refractory cancer includes disease that is progressing despite active treatment while “relapsed” cancer includes cancer that progresses in the absence of any current therapy, but following successful initial therapy. Accordingly, in embodiments, the subject is one who has undergone one or more previous regimens of therapy with one or more ‘standard-of-care’ therapeutic agents. In such cases, the subject's cancer may be considered refractory or relapsed.

A “subject” includes a mammal. The mammal can be e.g., any mammal, e.g., a human, primate, mouse, rat, dog, cat, cow, horse, goat, camel, sheep or a pig. Preferably, the mammal is a human. The term “patient” refers to a human subject.

As used herein, “treatment”, “treating” or “treat” describes the management and care of a patient for the purpose of combating a disease, condition, or disorder and includes the administration of apilimod to alleviate the symptoms or complications of a disease, condition or disorder, or to eliminate the disease, condition or disorder.

As used herein, “prevention”, “preventing” or “prevent” describes reducing or eliminating the onset of the symptoms or complications of the disease, condition or disorder and includes the administration of apilimod to reduce the onset, development or recurrence of symptoms of the disease, condition or disorder.

In one embodiment, the administration of a PIKfyve inhibitor leads to the elimination of a symptom or complication of the cancer being treated, however elimination of the cancer is not required. In one embodiment, the severity of the symptom is decreased. In the context of cancer, such symptoms may include clinical markers of severity or progression including the degree to which a tumor secretes growth factors, degrades the extracellular matrix, becomes vascularized, loses adhesion to juxtaposed tissues, or metastasizes, as well as the number of metastases.

Treating cancer according to the methods described herein can result in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer according to the methods described herein can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer according to the methods described herein can result in a decrease in the number of tumors. Preferably, after treatment, tumor number is reduced by 5% or greater relative to the number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer according to the methods described herein can result in a decrease in number of metastatic lesions in other tissues or organs distant from the primary tumor site. Preferably, after treatment, the number of metastatic lesions is reduced by 5% or greater relative to the number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer according to the methods described herein can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer according to the methods described herein can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer according to the methods described herein can result in increase in average survival time of a population of treated subjects in comparison to a population receiving monotherapy with a drug that is not apilimod. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer according to the methods described herein can result in a decrease in the mortality rate of a population of treated subjects in comparison to a population receiving carrier alone. Treating a disorder, disease or condition according to the methods described herein can result in a decrease in the mortality rate of a population of treated subjects in comparison to an untreated population. Treating a disorder, disease or condition according to the methods described herein can result in a decrease in the mortality rate of a population of treated subjects in comparison to a population receiving monotherapy with a drug that is not apilimod. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer according to the methods described herein can result in a decrease in tumor growth rate. Preferably, after treatment, tumor growth rate is reduced by at least 5% relative to the number prior to treatment; more preferably, tumor growth rate is reduced by at least 10%; more preferably, reduced by at least 20%; more preferably, reduced by at least 30%; more preferably, reduced by at least 40%; even more preferably, reduced by at least 50%; and most preferably, reduced by at least 75%. Tumor growth rate may be measured by any reproducible means of measurement. Tumor growth rate can be measured according to a change in tumor diameter per unit time. In one embodiment, after treatment, the tumor growth rate may be about zero and is determined to maintain the same size, e.g., the tumor has stopped growing.

Treating cancer according to the methods described herein can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by the failure of tumors to reoccur after treatment has stopped.

### Pharmaceutical Compositions and Formulations

The present disclosure provides pharmaceutical compositions comprising an amount of a PIKfyve inhibitor such as apilimod, or a pharmaceutically acceptable salt thereof, in combination with at least one pharmaceutically acceptable excipient or carrier, wherein the amount is effective for the treatment of a cancer as described herein, and/or effective to inhibit PIKfyve in the cancer cells of a subject having cancer. In embodiments, the pharmaceutically acceptable salt is selected from a sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salt. In embodiments, the pharmaceutically acceptable salt is selected from the group consisting of chloride, methanesulfonate, fumarate, lactate, maleate, pamoate, phosphate, and tartrate. In embodiments, the pharmaceutically acceptable salt is a dimesylate salt.

In one embodiment, the PIKfyve inhibitor is apilimod. In embodiments, the apilimod is apilimod free base. In one embodiment, the apilimod is apilimod dimesylate.

In embodiments, the PIKfyve inhibitor is combined with at least one additional active agent in a single dosage form. In one embodiment, the composition further comprises an antioxidant.

A “pharmaceutical composition” is a formulation containing the compounds described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). An effective amount of a pharmaceutical composition is that which provides an objectively identifiable improvement as noted by the clinician or other qualified observer. For example, alleviating a symptom of a disorder, disease or condition. As used herein, the term “dosage effective manner” refers to an amount of a pharmaceutical composition to produce the desired biological effect in a subject or cell.

For example, the dosage unit form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the disclosure may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition is in a form suitable for delivery by an intranasal or intrathecal route.

In embodiments, the pharmaceutical composition is in the form of an oral dosage form, for delivery perorally. In embodiments, the composition is in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present disclosure with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present disclosure may be suspended or dissolved in an oily phase and combined with emulsifying and/or suspending agents. If desired, certain sweetening and/or flavoring and/or coloring agents may be added.

In embodiments, the pharmaceutical composition is in the form of a tablet. The tablet can comprise a unit dosage of a compound of the present disclosure together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methylcellulose, ethylcellulose, hydroxypropyl methylcellulose, and starches such as corn starch. The tablet can further comprise binding and granulating agents such as polyvinylpyrrolidone, disintegrants (e.g., swellable crosslinked polymers such as crosslinked carboxymethylcellulose), lubricating agents (e.g., stearates), preservatives (e.g., parabens), antioxidants (e.g., BHT), buffering agents (for example phosphate or citrate buffers), and effervescent agents such as citrate/bicarbonate mixtures.

The tablet can be a coated tablet. The coating can be a protective film coating (e.g., a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present disclosure may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present disclosure as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present disclosure can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the disclosure are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present disclosure also provides packaging and kits comprising pharmaceutical compositions for use in the methods of the present disclosure. The kit can comprise one or more containers selected from the group consisting of a bottle, a vial, an ampoule, a blister pack, and a syringe. The kit can further include one or more of instructions for use in treating and/or preventing a disease, condition or disorder of the present disclosure, one or more syringes, one or more applicators, or a sterile solution suitable for reconstituting a pharmaceutical composition of the present disclosure.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

### Example 1: Mutation in RAS Pathway Renders Cells Sensitive to Apilimod

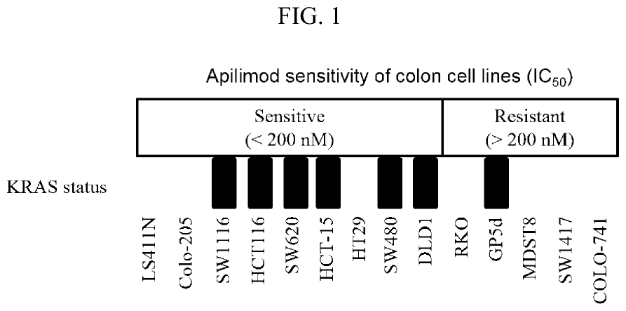

In a sensitivity screen in 146 cancer cell lines we found that colorectal cancer was the second most sensitive type of cancer to apilimod. Nine out of 14 colorectal cancer cell lines screened (64%) were sensitive to apilimod, where a cell line was defined as sensitive if it had an average IC₅₀ of less than 200 nM in at least two independent 5-day viability assays using the CellTiter-Glo™ assay system (Promega). Activating Ras pathway mutations are a frequent occurrence in colorectal cancer. For example, activating KRAS mutations occur in about 30-50% of patients. Since this is a known ‘escape’ pathway for cancer cells, we assessed whether the difference in sensitivity to apilimod among the colorectal cancer cell lines was related to KRAS mutation status. Surprisingly, apilimod-sensitive colorectal cancer lines were enriched in KRAS activating mutations (FIG. 1). Each of the SW1116, HCT116, SW620, HCT-15, SW480, and DLD-1 cell lines, representing 6 out of the 9 sensitive cell lines, carries an activating RAS mutation, detailed in Table 1 below. GP5d was the only cell line that harbors a KRAS mutation, detailed in Table 1, that was not defined as sensitive to apilimod based on the cut-off of 200 nM, although the IC₅₀ was 209 nM (see Table 2). Each of these mutations promotes constitutively active KRAS by preventing GTP hydrolysis.

TABLE 1
KRAS mutations in colorectal adenocarcinoma cancer cell lines

| Cell Line | Mutation |
|---|---|
| SW1116 | p.G12A |
| HCT116 | p.G13D |
| SW620 | p.G12V |
| HCT-15 | p.G13D |
| SW480 | p.G12V |
| DLD-1 | p.G13D |
| GP5d | p.G12D |

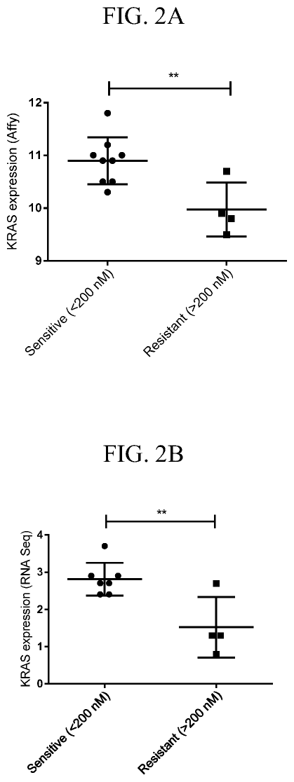

Since elevated expression of KRAS can also result in constitutive activity, we also assessed whether there was an association between KRAS expression levels in these cells and apilimod sensitivity. The KRAS expression level for each of the cell lines was obtained from the Cancer Cell Line Encyclopedia (CCLE) database. Two measures of KRAS mRNA expression are shown in FIG. 2. Panel A shows expression as determined by Affymetrix microarray and Panel B shows expression as determined by RNA seq analysis. In both cases, expression is log2-normalized. FIG. 2A-B shows a positive association between apilimod sensitivity and high KRAS expression in this panel of cell lines, consistent with the association seen between apilimod sensitivity and activating KRAS mutations.

These results indicate that apilimod sensitive colorectal cancer cell lines are characterized by enhanced activation of KRAS signaling, which may be due either to the presence of activating KRAS mutations or up-regulated KRAS expression. The molecular mechanisms of the enhanced sensitivity to apilimod seen in these experiments is unknown, but we believe it may be related to the ability of apilimod, as an inhibitor of PIKfyve, to inhibit autophagy, which we recently demonstrated, see Gayle et al., (2017) Blood 129(13), 1768-1778 and Gayle et al., (2017) Autophagy 13(6), 1082-1083. Autophagy is one mechanism by which cancer cells may enhance their survival. Without being bound by any theory, the sensitivity to apilimod observed in this study may be due, in part, to the reliance of the cancer cells on autophagy for their survival in the presence of constitutively active Ras pathway signaling. If this is the case, then cancer cells characterized by activated Ras pathway signaling may be particularly susceptible to combination therapy with a PIKfyve inhibitor and a Ras pathway inhibitor. We tested this in the following example.

### Example 2: Apilimod Displays Synergistic Activity with RAS Pathway Inhibitors

Having demonstrated that cell lines harboring mutations in the RAS signaling pathway are sensitive to apilimod single agent, we assessed whether apilimod can work synergistically with RAS pathway inhibitors in cancer cells characterized by constitutively active RAS pathway signaling. The cell lines used in this study were derived from lung cancers, colorectal cancers, pancreatic cancer and melanoma, as shown in Table 2 below. The table also shows the average IC₅₀ for apilimod alone, as determined from two independent experiments. The calculation of the IC₅₀ corresponding to single agent activity was performed using the R package DRC package (Ritz et al., (2015) PLoS One 10(12), e0146021.doi.10.1371/journal.pone.0146021; Team R, (2017) J. Open Statistics 7(5).

TABLE 2
Cell lines used in Example 2 and apilimod IC₅₀ (single agent)

| | | | | Average |
|---|---|---|---|---|
| Cancer | | | | Apilimod |
| type | Cancer Subtype | Cell line | Mutation | IC₅₀ (nM) |
| Lung | NSCLC | A549 | KRAS G12S | 49 |
| | NSCLC | NCI-H1944 | KRAS G13D | 36 |
| | NSCLC | NCI-H2030 | KRAS G12C | 228 |
| | NSCLC | NCI-H1792 | KRAS G12C | >400 |
| | NSCLC | NCI-H2122 | KRAS G12C | >400 |
| | NSCLC | A427 | KRAS G12D | 80 |
| Colorectal | Adenocarcinoma, | GP5d | KRAS G12D | 209 |
| | Dukes' B | | | |
| | Adenocarcinoma, | LS411N | BRAF V600E | 89 |
| | Dukes' B | | | |
| | Adenocarcinoma, | HCT-15 | KRAS G13D | 47 |
| | Dukes' C | | | |
| | Adenocarcinoma | HT-29 | BRAF V600E | 90 |
| | Adenocarcinoma | RKO | BRAF V600E | 294 |
| Pancreatic | Adenocarcinoma | BxPC3 | BRAF | >400 |
| | | | delV487-P492 | |
| Melanoma | Amelanotic | A375 | BRAF V600E | 104 |

NSCLC = Non-small cell lung cancer.

For assessment of synergistic activity, cells of each cell line were seeded at optimal density for growth onto different 96-well plates and placed into a humidified incubator (37° C., 5% CO₂) to adhere overnight. The following day, the cells on each plate were treated either with a single agent (a PIKfyve inhibitor, e.g., apilimod, or a RAS pathway inhibitor) or with a combination of the two agents. Cells were treated for 120 hours and then assayed for cell viability using CellTiter-Glo™ (Promega). Synergistic activity was assessed using the combination index (CI) values calculated according to the Chou-Talalay method as described in Chou T-C, (2010) Cancer Res. 70(2), 440-446.

Using this method, a CI value is calculated based on the effects of each drug alone on cell viability (single agent treatment) and the effects of each drug combination, across all combinations of both agents tested. The single agent treatments consisted of 8 different concentrations of each single agent, obtained by serial dilution. The concentrations were chosen to provide a range above and below the IC₅₀ value of the single agent (if achieved) in a given cell line. For example, the IC₅₀ of apilimod in LS411N cells was determined to be 89 nM, and the 8 different concentrations tested were 23.4, 35.1, 52.7, 79.0, 119, 178, 267, and 400 nM (values rounded to 3 significant digits). Table 3 shows the concentration ranges used for each of the agents in the cell lines tested, which are shown in the summary table, Table 5. The concentration ranges shown in Table 3 were suitable for all cell lines tested, except where a second concentration range is indicated. For example, for the combination of apilimod and trametinib, two different trametinib concentration ranges were utilized, the first range shown in Table 4 below was used in GP5d cells and the second range was used in the other cell lines (see Table 5 for detail).

TABLE 3
Agents and concentration ranges used for assessment of
synergistic activity.

| Target | Agent | Concentration range (fold dilution) |
|---|---|---|
| PIKfyve | Apilimod | 23-400 nM (1.5-fold) |
| MEK | Trametinib | 39-5000 nM (2-fold); |
| | | 2-250 nM (2-fold) |
| | Selumetinib | 78-10000 nM (2-fold) |
| | Cobimetinib | 39-5000 nM (2-fold) |
| | Binimetinib | 39-5000 nM (2-fold) |
| BRAF V600E | Vemurafenib | 78-10000 nM (2-fold) |
| RAF-1, BRAF, | Regorafenib | 78-10000 nM (2-fold) |
| BRAF V600E | | |
| ERK | BVD-523 | 39-5000 nM (2-fold) |

In each experiment for a given cell line, the combination treatments consisted of 8×8 or 64 different combination treatments. To illustrate the method, Table 4A shows normalized cell viability values obtained using the CellTiter-Glo™ assay for each single agent treatment (apilimod or selumetinib) in RKO cells.

TABLE 4A
Normalized cell viability values for single agent (apilimod or
selumetinib) in RKO cells.

| Apilimod (nM) | | Selumetinib (nM) | |
|---|---|---|---|
| (single agent) | Viability | (single agent) | Viability |
| 400 | 0.44 | 100000 | 0.32 |
| 267 | 0.53 | 5000 | 0.46 |
| 178 | 0.70 | 2500 | 0.53 |
| 119 | 0.97 | 1250 | 0.64 |
| 79 | 0.93 | 625 | 0.74 |
| 53 | 0.96 | 313 | 0.71 |
| 35 | 0.93 | 125 | 0.78 |
| 23 | 0.98 | 78 | 0.78 |

Table 4B shows normalized cell viability for each of the 64 combination treatments of apilimod and selumetinib in the same cells.

TABLE 4B
Normalized cell viability for the 64 combinations of apilimod and selumetinib in
RKO cells.

| | | 8x8 = 64 combinations | | | | | | | |
|---|---|---|---|---|---|---|---|---|---|
| Apilimod | 400 | 0.31 | 0.25 | 0.16 | 0.14 | 0.13 | 0.12 | 0.10 | 0.11 |
| | 267 | 0.41 | 0.31 | 0.23 | 0.20 | 0.17 | 0.14 | 0.13 | 0.13 |
| | 178 | 0.53 | 0.43 | 0.33 | 0.25 | 0.24 | 0.19 | 0.17 | 0.15 |
| | 119 | 0.78 | 0.68 | 0.50 | 0.42 | 0.36 | 0.32 | 0.25 | 0.24 |
| | 79 | 0.94 | 0.80 | 0.77 | 0.70 | 0.60 | 0.47 | 0.40 | 0.35 |
| | 53 | 0.93 | 0.83 | 0.77 | 0.70 | 0.70 | 0.60 | 0.50 | 0.40 |
| | 35 | 0.94 | 0.82 | 0.80 | 0.77 | 0.70 | 0.63 | 0.57 | 0.42 |
| | 23 | 0.92 | 0.84 | 0.79 | 0.81 | 0.75 | 0.67 | 0.63 | 0.43 |
| | | 78 | 156 | 313 | 625 | 1250 | 2500 | 5000 | 10000 |

Selumetinib (nM)

Table 4C shows the CI values obtained for each of the 64 combination treatments of apilimod and selumetinib in these cells. Using this method, combinations with CI values>1 are considered antagonistic, CI values=1 are considered additive, and CI values<1 are considered synergistic. CI values associated with a reduction of cell viability to less than or equal to 25% are shown in bold.

TABLE 4C
Combination Index (CI) values for the 64 (8x8) combination treatments of
apilimod and selumetinib in RKO cells.

| | | | | | | | | | |
|---|---|---|---|---|---|---|---|---|---|
| Apilimod | 400 | 0.92 | 0.80 | 0.64 | 0.60 | 0.58 | 0.55 | 0.51 | 0.55 |
| (nM) | 267 | 0.74 | 0.61 | 0.51 | 0.47 | 0.45 | 0.41 | 0.41 | 0.41 |
| | 178 | 0.62 | 0.53 | 0.44 | 0.37 | 0.37 | 0.33 | 0.33 | 0.33 |
| | 119 | 1.16 | 0.44 | 0.47 | 0.42 | 0.39 | 0.40 | 0.36 | 0.45 |
| | 79 | >2 | 0.47 | >2 | 1.83 | 1.33 | 0.86 | 0.85 | 0.93 |
| | 53 | >2 | >2 | >2 | 1.65 | >2 | >2 | 1.76 | 1.38 |
| | 35 | >2 | >2 | >2 | >2 | >2 | >2 | >2 | 1.51 |
| | 23 | >2 | >2 | >2 | >2 | >2 | >2 | >2 | 1.76 |
| | | 78 | 156 | 313 | 625 | 1250 | 2500 | 5000 | 10000 |

Selumetinib (nM)

In brief, CI was defined mathematically as follows:

$$CI = \frac{D 1}{D 1 a l o n e} + \frac{D 2}{D 2 a l o n e} \qquad ( Eq \text{-} 2 )$$

with:
D1 and D2 being the doses of Drug 1 and Drug 2 in the combination treatment, respectively, that give viability, V.
D1 alone and D2 alone being the doses of Drug 1 and Drug 2, respectively, as a single agent that would give the same viability V as that of the combination. D1 alone and D2 alone were estimated from Hill's equation:

$$Dalone = EC 50 * {( \frac{1 - V}{V} )}^{\frac{1}{Hill}} \qquad ( Eq \text{-} 3 )$$

with EC₅₀ in Equation 3 corresponding to IC₅₀ in our experiments, and the Hill slope corresponding to the Drug 1 or Drug 2 fitted viability curve.

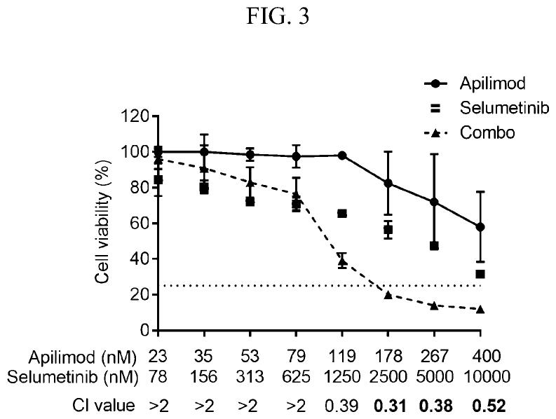

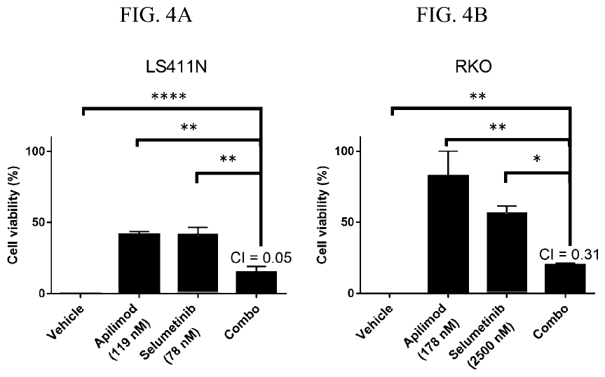

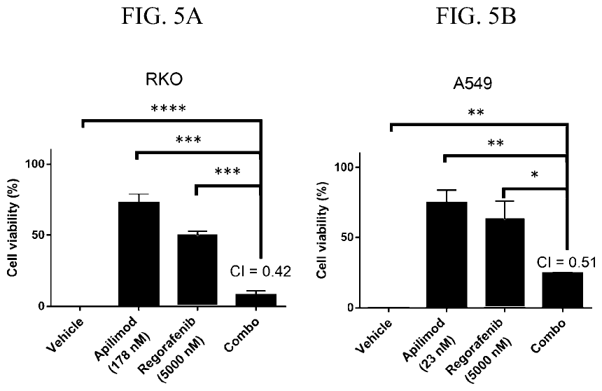

For illustration, FIG. 3 shows dose response curves (each point is an average of two independent experiments) for each of the 8 doses of each single agent treatment, apilimod or selumetinib, and 8 of the combination doses representing the combination of each single agent doses. The CI values for each dose combination are also shown and CI values associated with a reduction of cell viability to less than or equal to 25% are shown in bold. Reduced viability of the combination versus the single agents is observed at combinations producing CI values<1, i.e., 119 nM apilimod/ 1250 nM selumetinib, 178 nM apilimod/2500 nM selumetinib, 267 apilimod/5000 nM selumetinib, and 400 nM apilimod/10,000 nM selumetinib. The best synergy between these two agents, as indicated by the lowest average CI value of 0.31, was obtained using 178 nM apilimod and 2500 nM selumetinib (rounded to 3 significant digits). FIG. 4 provides a graphical representation of this ‘best synergy’ data for apilimod and selumetinib in LS411N cells (Panel A) and RKO cells (Panel B), illustrating the effects of the combination at these optimal doses, compared to single agent treatment at the same dose. Statistical significance of the optimal combination dose compared to single agent doses was determined using one-way ANOVA, Dunnett's multiple comparisons test. FIG. 5 provides the same graphical representation of this ‘best synergy’ data for apilimod and regorafenib in RKO cells (Panel A) and A549 cells (Panel B).

We performed this analysis using apilimod and the RAS pathway inhibitors trametinib, selumetinib, cobimetinib, binimetinib, BVD-523, vemurafenib and regorafenib. For each cell line, agent, and set of 64 dose combinations, we conducted two independent experiments. Table 5 provides a summary of the results obtained from these experiments. In the table, the combination dose that gave the best synergy (lowest average CI value) for each combination of agents in each cell line is shown, along with the average CI value.

TABLE 5
Summary of synergism between apilimod and RAS pathway inhibitors
in different cell lines harboring mutations in RAS or BRAF.

| | Apilimod | RAS pathway inhibitor/ | | | Average |
|---|---|---|---|---|---|
| | concentration | concentration | | | Combination |
| Cell line | (nM) | (nM)/IC₅₀ (nM) | | | Index value |
| GP5d | 200 | Trametinib | 39 | >5000 | 0.51 |
| GP5d | 200 | Cobimetinib | 312.5 | >5000 | 0.5 |
| GP5d | 200 | Binimetinib | 78 | >5000 | 0.48 |
| A549 | 50 | Cobimetinib | 156 | 403 | 0.53 |
| A549 | 35 | Trametinib | 31 | 17 | 0.21 |
| A549 | 23 | Regorafenib | 5000 | 6376 | 0.51 |
| A427 | 119 | Vemurafenib | 10000 | >10000 | 0.61 |
| NCI-H1944 | 79 | Vemurafenib | 2500 | 6835 | 0.38 |
| NCI-H1792 | 178 | Trametinib | 63 | 33 | 0.51 |
| NCI-H1792 | 267 | Vemurafenib | 10000 | >10000 | 0.77 |
| NCI-H2030 | 178 | Vemurafenib | 10000 | >10000 | 0.81 |
| NCI-H2122 | 119 | Vemurafenib | 10000 | >10000 | 0.17 |
| LS411N | 53 | Trametinib | 6.3 | 1 | 0.3 |
| LS411N | 119 | Selumetinib | 78 | 46 | 0.05 |
| LS411N | 119 | Vemurafenib | 1250 | 350 | 0.17 |
| HCT-15 | 79 | Vemurafenib | 10000 | >10000 | 0.55 |
| HCT-15 | 35 | Regorafenib | 2500 | 7054 | 0.51 |
| HT-29 | 178 | Trametinib | 25 | 3 | 0.08 |
| HT-29 | 178 | Vemurafenib | 2500 | 2466 | 0.24 |
| HT-29 | 35 | Regorafenib | 5000 | 6513 | 0.49 |
| RKO | 178 | Trametinib | 31 | 32 | 0.32 |
| RKO | 178 | Selumetinib | 2500 | 3507 | 0.31 |
| RKO | 178 | Vemurafenib | 5000 | 7116 | 0.12 |
| RKO | 178 | Regorafenib | 5000 | 4917 | 0.42 |
| RKO | 178 | BVD-523 | 1250 | 945 | 0.6 |
| A375 | 53 | Trametinib | 6.3 | 2 | 0.48 |
| A375 | 79 | Vemurafenib | 1250 | 1328 | 0.42 |

Average IC₅₀ and CI values determined from two independent experiments.

In summary, these experiments demonstrated that apilimod is synergistic (CI<1) with the MEK inhibitors trametinib, selumetinib, cobimetinib and binimetinib, and with ERK inhibitor BVD-523, as well as with the BRAF inhibitors, vemurafenib and regorafenib (Table 5). These data further show that apilimod is synergistic with RAS pathway inhibitors in cell lines harboring RAS or BRAF mutations and suggest that PIKfyve inhibitors, perhaps as inhibitors of autophagy, provide a novel therapeutic regimen for combination therapy with RAS pathway inhibitors in treating cancers characterized by activation of RAS pathway signaling.

### Example 3: Cancer Cells with MET Pathway Activation are Sensitive to Apilimod

We also found that cell lines harboring MET pathway activation via amplification, mutation or autocrine signaling were sensitive to apilimod. Out of ten cell lines harboring MET pathway activation we found that 60% (6/10) of the cell lines were sensitive to apilimod with IC₅₀ less than 200 nM in a 5-day viability assay (Table 6).

TABLE 6
Cell lines harboring MET pathway activation display
apilimod sensitivity.

| | Cancer | | MET Pathway | Average |
|---|---|---|---|---|
| Cancer type | subtype | Cell line | Activation | IC₅₀ |
| Lung | NSCLC | NCI-H820 | AMP | 103 |
| | SCLC | SBC-5 | AMP | 108 |
| | NSCLC | EBC-1 | AMP | 113 |
| | NSCLC | NCI-H596 | Exon 14 | 304 |
| | NSCLC | NCI-H1993 | AMP | 877 |
| | NSCLC | NCI-H2023 | Autocrine | 73 |
| Gastric | Diffuse | Hs746T | AMP, Exon 14 | 108 |
| | Diffuse | MKN45 | AMP | 191 |
| | Diffuse | SNU-5 | AMP | 862 |
| Renal | Clear cell | Caki-1 | AMP, V1220I | 373 |

*NSCLC = Non-small cell lung cancer, SCLC = Small cell lung cancer, AMP = amplification, Exon 14 = exon 14 exon skipping activating mutation, Autocrine (Baltschukat et al., (2019) Clin Cancer Res 25(10), 3164-3175).
Average IC₅₀ determined from two independent experiments.

### Example 4: Apilimod Displays Synergistic Activity with MET and RAS Pathway Inhibitors in Cells with MET Pathway Activation

For assessment of synergistic activity of apilimod with MET inhibitors, we utilized the same methods as described above in Example 2. Here, we utilized cells harboring MET pathway activation, particularly the EBC-1, MKN45 and Caki-1 cell lines. In addition to MET inhibitors, since MET activates the RAS pathway, we also assayed for synergistic activity with inhibitors of the RAS pathway in these cells. Table 7 provides the concentration ranges used for each agent. The concentration ranges shown in Table 7 were suitable for all cell lines tested, except in those cases where multiple concentration ranges were tested. For example, for each of the combinations of apilimod and the MET inhibitors crizotinib, cabozantinib, tepotinib and AMG 337, two concentration ranges were tested for synergistic activity with apilimod. Multiple concentration ranges were also tested for each of the combinations of apilimod with the MEK inhibitor selumetinib and the ERK inhibitor GDC-0994.

TABLE 7
List of drugs and concentrations used for drug screening of
synergistic activity.

| | | Concentration range |
|---|---|---|
| Target | Drug | (fold dilution) |
| PIKfyve | Apilimod | 23-400 nM (1.5-fold) |
| | | 3-400 nM (2-fold) |
| | APY-0201 | 23-400 nM (1.5-fold) |
| MET | Crizotinib | 0.4-50 nM (2-fold) |
| | | 3-50 nM (1.5 fold) |
| | Capmatinib | 0.1-12.5 nM (2-fold) |
| | Cabozantinib | 0.4-50 nM (2-fold) |
| | | 6-100 nM (1.5 fold) |
| | Tepotinib | 0.4-50 nM (2-fold) |
| | | 1.5-25 nM (1.5-fold) |
| | AMG 337 | 0.4-50 nM (2-fold) |
| | | 1.5-25 nM (1.5-fold) |
| | Savolitinib | 0.1-12.5 nM (2-fold) |
| MEK | Trametinib | 2-250 nM (2-fold) |
| | Selumetinib | 8-1000 nM (2-fold) |
| | | 78-10000 nM (2-fold) |
| | Binimetinib | 39-5000 nM (2-fold) |
| | Cobimetinib | 39-5000 nM (2-fold) |
| ERK | GDC-0994 | 39-5000 nM (2-fold) |
| | | 156-20000 nM (2-fold) |
| | BVD-523 | 39-5000 nM (2-fold) |
| RAF-1, BRAF, | Regorafenib | 78-10000 nM (2-fold) |
| BRAF V600E | | |

Tables 8-11 provide a summary of the results obtained from these experiments showing the combination dose that gave the best synergy (lowest average CI value) for each combination of agents in each cell line, along with the average CI value.

In the MET-amplified lung cancer cell line EBC-1, apilimod was synergistic with the MET inhibitors crizotinib, capmatinib, cabozantinib, tepotinib, AMG 337 and savolitinib (Table 8), the MEK inhibitors trametinib, selumetinib, binimetinib and cobimetinib (Table 9), and the ERK inhibitors BVD-523 and GDC-0994 (Table 9).

Synergy was also observed in the MET-amplified gastric cell line MKN45 with crizotinib, capmatinib, cabozantinib, tepotinib, AMG 337, and savolitinib (Table 8), the MEK inhibitors trametinib, selumetinib, binimetinib and cobimetinib (Table 9), and the ERK inhibitors BVD-523 and GDC-0994 (Table 9).

The Caki-1 cells are resistant to MET inhibitors, but we found synergy with apilimod and the MEK inhibitors trametinib and cobimetinib, as well as with the RAF inhibitor regorafenib (Table 9).

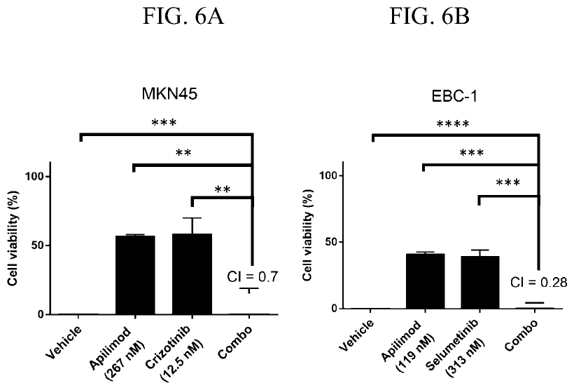

FIG. 6 provides a graphical representation of the ‘best synergy’ data for apilimod and crizotinib in MKN45 cells (Panel A) and apilimod and selumetinib in EBC-1 cells (Panel B), illustrating the effects of the combination at these optimal doses, compared to single agent treatment at the same dose. Statistical significance of the optimal combination dose compared to single agent doses was determined using one-way ANOVA, Dunnett's multiple comparisons test.

These data demonstrate that apilimod displays synergistic activity when combined with MET or RAS pathway inhibitors in cells harboring activated MET.

TABLE 8
Summary of synergism between apilimod and MET pathway
inhibitors in MET- activated cell lines. Average IC₅₀ and CI
values determined from two independent experiments.

| | Apilimod | MET pathway inhibitor/ | | | Average |
|---|---|---|---|---|---|
| | concentration | concentration | | | Combination |
| Cell line | (nM) | (nM)/IC₅₀ (nM) | | | Index value |
| EBC-1 | 119 | Crizotinib | 12.5 | 10 | 0.63 |
| EBC-1 | 200 | Capmatinib | 0.8 | 0.7 | 0.51 |
| EBC-1 | 119 | Cabozantinib | 25 | 28 | 0.78 |
| EBC-1 | 200 | Tepotinib | 6.3 | 13 | 0.85 |
| EBC-1 | 200 | AMG 337 | 3 | 4 | 0.51 |
| EBC-1 | 119 | Savolitinib | 2 | 1.5 | 0.65 |
| MKN45 | 267 | Crizotinib | 12.5 | 13 | 0.7 |
| MKN45 | 178 | Capmatinib | 2 | 1.5 | 0.88 |
| MKN45 | 267 | Cabozantinib | 67 | 67 | 0.87 |
| MKN45 | 178 | Tepotinib | 17 | 16 | 0.81 |
| MKN45 | 178 | AMG 337 | 7 | 7 | 0.8 |
| MKN45 | 178 | Savolitinib | 3 | 3 | 0.8 |

TABLE 9
Summary of synergism between apilimod and RAS pathway
inhibitors in MET-activated cell lines. Average IC₅₀ and CI values
determined from two independent experiments.

| | Apilimod | | | | Average |
|---|---|---|---|---|---|
| | concentration | RAS pathway inhibitor/ | | | Combination |
| Cell line | (nM) | concentration (nM)/IC₅₀ (nM) | | | Index value |
| EBC-1 | 200 | Trametinib | 8 | 10 | 0.19 |
| EBC-1 | 119 | Selumetinib | 313 | 160 | 0.28 |
| EBC-1 | 119 | Binimetinib | 156 | 75 | 0.19 |
| EBC-1 | 119 | Cobimetinib | 156 | 136 | 0.42 |
| EBC-1 | 119 | BVD-523 | 1250 | 866 | 0.55 |
| EBC-1 | 79 | GDC-0994 | 5000 | 4152 | 0.5 |
| MKN45 | 178 | Trametinib | 16 | 18 | 0.4 |
| MKN45 | 178 | Selumetinib | 1250 | 1107 | 0.22 |
| MKN45 | 178 | Binimetinib | 313 | 533 | 0.23 |
| MKN45 | 178 | Cobimetinib | 156 | 194 | 0.57 |
| MKN45 | 119 | BVD-523 | 625 | 433 | 0.47 |
| MKN45 | 178 | GDC-0994 | 5000 | 5331 | 0.62 |
| Caki-1 | 178 | Trametinib | 31 | 47 | 0.34 |
| Caki-1 | 178 | Cobimetinib | 5000 | >5000 | 0.02 |
| Caki-1 | 178 | Regorafenib | 5000 | 5955 | 0.49 |

In addition, using the same CI analysis discussed above, we tested an alternative PIKfyve inhibitor, APY-0201, to assess whether the synergistic activity was generalizable from apilimod to other inhibitors of PIKfyve. In these experiments, summarized in Tables 10 and 11 below, APY-0201 was synergistic with crizotinib and trametinib in both the EBC-1 and MKN45 cell lines. These data indicate that the synergistic activity is due to on-target inhibition of PIKfyve.

TABLE 10
Summary of synergism between APY-0201 and MET pathway
inhibitor Crizotinib.

| | APY-0201 | | MET pathway | | | Average |
|---|---|---|---|---|---|---|
| | concentration | | inhibitor/concentration | | | Combination |
| Cell line | (nM)/IC₅₀ (nM) | | (nM)/IC₅₀ (nM) | | | Index value |
| EBC-1 | 178 | 171 | Crizotinib | 12.5 | 14 | 0.76 |
| MKN45 | 178 | 269 | Crizotinib | 22 | 23 | 0.84 |

Average IC₅₀ and CI values determined from two independent experiments.

Average IC₅₀ and CI values determined from two independent experiments.

TABLE 11
Summary of synergism between APY-0201 and RAS pathway
inhibitor Trametinib.

| | APY-0201 | | RAS pathway | | | Average |
|---|---|---|---|---|---|---|
| | concentration | | inhibitor/concentration | | | Combination |
| Cell line | (nM)/IC₅₀ (nM) | | (nM)/IC₅₀ (nM) | | | Index value |
| EBC-1 | 178 | 171 | Trametinib | 16 | 7 | 0.32 |
| MKN45 | 178 | 269 | Trametinib | 16 | 13 | 0.31 |

Average IC₅₀ and CI values determined from two independent experiments.

Average IC₅₀ and CI values determined from two independent experiments.

What is claimed is:

1. A method for treating a cancer associated with activated MET or RAS pathway signaling in a subject in need thereof, the method comprising administering to the subject a pharmaceutical composition comprising a PIKfyve inhibitor, alone or in combination with a MET inhibitor or a RAS pathway inhibitor.

2. A method for treating a cancer in a subject in need thereof, the method comprising determining, ex vivo, the presence of a biomarker of activated MET or RAS pathway signaling in a biological sample comprising cancer cells from the subject, and administering to the subject whose cancer cells are positive for the biomarker a pharmaceutical composition comprising a PIKfyve inhibitor, alone or in combination with a MET inhibitor or a RAS pathway inhibitor.

3. The method of claim 1, wherein the PIKfyve inhibitor is selected from YM201636, WX8(MLS000543798), NDF(MLS000699212), WWL(MLS000703078), XB6(MLS001167897), XBA(MLS001167909), Vacuolin-1, APY-0201, and apilimod, and pharmaceutically acceptable salts thereof.

4. The method of claim 3, wherein the PIKfyve inhibitor is apilimod, or a pharmaceutically acceptable salt thereof

5. The method of claim 1, wherein the cancer is a carcinoma, a sarcoma, or a glioma.

6. The method of claim 5, wherein the cancer cells contain an activating mutation in the RAS or MET pathway.

7. The method of claim 5, wherein the cancer is selected from appendiceal cancer, bladder cancer, brain cancer, breast cancer, cervical cancer, colorectal cancer, esophageal cancer, gastric cancer, gastrointestinal carcinoma, gastrointestinal stromal tumor (GIST), genitourinary cancer, glioma, head and neck cancer, hepatocellular carcinoma, lung cancer, melanoma, mesothelioma, non-small cell lung cancer, ovarian cancer, pancreatic cancer, prostate cancer, renal cell carcinoma, sarcoma, small cell lung cancer, soft tissue sarcoma, testicular cancer, thyroid tumor, and uterine carcinosarcoma.

8. The method of claim 5, wherein the carcinoma is selected from adenocarcinoma, basal cell carcinoma, squamous cell carcinoma, transitional cell carcinoma, large cell carcinoma, and melanoma.

9. The method of claim 5, wherein the carcinoma is selected from a pancreatic ductal adenocarcinoma (PDAC), a colorectal carcinoma, a lung carcinoma, such as a non-small cell lung cancer (NSCLC), a renal carcinoma, a head and neck cancer, such as a head and neck squamous cell carcinoma (HNSCC), a gastric carcinoma (GC), and a hepatocellular carcinoma (HCC).

10. The method of claim 5, wherein the sarcoma is a soft tissue sarcoma, such as a gastrointestinal stromal tumor (GIST), or a uterine carcinosarcoma.

11. The method of claim 1, wherein the pharmaceutical composition comprising the PIKfyve inhibitor is administered in combination with a MET inhibitor or a RAS pathway inhibitor.

12. The method of claim 11, wherein the PIKfyve inhibitor is administered in the same composition or in a different composition from the MET or RAS pathway inhibitor.

13. The method of claim 11, wherein the MET pathway inhibitor is selected from crizotinib, capmatinib, tepotinib, AMG337, cabozantinib, savolitinib (AZD6094, HMPL-504), tivantinib, foretinib, volitinib, SU11274, PHA 665752, SGX523, BAY-853474, KRC-408, T-1840383, MK-2461, BMS-777607, JNJ-38877605, tivantinib (ARQ 197), PF-04217903, MGCD265, BMS-754807, BMS-794833, AMG-458, NVP-BVU972, AMG-208, golvatinib, norcantharidin, S49076, SAR125844, merestinib (LY2801653), onartuzumab, emibetuzumab, SAIT301, ABT-700, DN30, LY3164530, rilotumumab, ficlatuzumab, TAK701, and YYB-101.

14. The method of claim 13, wherein the MET inhibitor is selected from crizotinib, capmatinib, tepotinib, AMG337, cabozantinib, and savolitinib (AZD6094, HMPL-504).

15. The method of claim 13, wherein the cancer cells contain an activating mutation in the MET pathway.

16. The method of any one of claims 13, wherein the cancer is a carcinoma, a glioma, or a sarcoma.

17. The method of claim 16, wherein the cancer is a carcinoma.

18. The method of claim 17, wherein the carcinoma is selected from breast cancer, colorectal cancer, esophageal cancer, gastric cancer, liver cancer, lung cancer, and renal cancer.

19. The method of claim 18, wherein the carcinoma is selected from lung cancer, gastric cancer, and renal cancer.

20. The method of claim 19, wherein the lung cancer is a small cell lung cancer (SCLC) or a non-small cell lung cancer (NSCLC).

21. The method of claim 16, wherein the cancer is a soft tissue sarcoma, such as a gastrointestinal stromal tumor (GIST), or a uterine carcinosarcoma.

22. The method of claim 11, wherein the RAS pathway inhibitor is selected from BVD-523, GDC-0994, binimetinib, cobimetinib, regorafenib, selumetinib, trametinib, vemurafenib, ARS1620, AMG510, AZD4785, MRTX1257, MRTX849, PD-0325901, dabrafenib, encorafenib, pimasertib, and sorafenib.

23. The method of claim 22, wherein the RAS pathway inhibitor is selected from BVD-523, GDC-0994, trametinib, cobimetinib, binimetinib, selumetinib, regorafenib and vemurafenib.

24. The method of claim 22, wherein the cancer cells contain an activating mutation in the RAS pathway.

25. The method of claim 22, wherein the cancer is selected from a carcinoma, a glioma, or a sarcoma.

26. The method of claim 25, wherein the cancer is selected from appendiceal cancer, bladder cancer, brain cancer, breast cancer, cervical cancer, colorectal cancer, esophageal cancer, gastric cancer, gastrointestinal carcinoma, gastrointestinal stromal tumor (GIST), genitourinary cancer, glioma, head and neck cancer, hepatocellular carcinoma, lung cancer, melanoma, mesothelioma, non-small cell lung cancer, ovarian cancer, pancreatic cancer, prostate cancer, renal cell carcinoma, sarcoma, small cell lung cancer, soft tissue sarcoma, testicular cancer, thyroid tumor, and uterine carcinosarcoma.

27. The method of claim 25, wherein the cancer is a carcinoma selected from bladder cancer, cervical cancer, colorectal cancer, gastric cancer, head and neck squamous cell carcinoma, lung cancer, melanoma, pancreatic cancer, prostate cancer, thyroid cancer, uterine cancer, and urothelial cancer.

28. The method of claim 27, wherein the cancer is selected from a colorectal cancer, a lung cancer, a melanoma, and a pancreatic cancer.

29. The method of claim 28, wherein the lung cancer is a small cell lung cancer (SCLC) or a non-small cell lung cancer (NSCLC).

30. The method of claim 2, wherein the biomarker of activated MET or RAS pathway signaling is selected from amplification of c-MET, an activating mutation in exon 14 of c-MET, an activating KRAS, NRAS or HRAS mutation and an activating BRAF mutation.

31. The method of claim 1, wherein the cancer is refractory to standard treatment, or wherein the cancer is metastatic.

32. The method of claim 2, wherein the step of determining, ex vivo, the presence of the biomarker comprises a polymerase chain reaction (PCR)-based assay, 5′exonuclease fluorescence assay, sequencing-by-probe hybridization, dot blotting, oligonucleotide array hybridization analysis, dynamic allele-specific hybridization, molecular beacons, restriction fragment length polymorphism (RFLP)-based methods, flap endonuclease-based methods, primer extension, 5′-nuclease-based methods, oligonucleotide ligase assays, single-stranded conformation polymorphism assays (SSCP), temperature gradient gel electrophoresis, denaturing high performance liquid chromatography (HPLC), high-resolution melting analysis, DNA mismatch-binding methods, capillary electrophoresis, fluorescence in situ hybridization (FISH) and next-generation sequencing (NGS) methods, or a combination of any of the foregoing.
